SEQUENCE LISTING (8 sequences)

SEQ ID NO: 1 - protein, 320 aa, synthetic construct
  LIPID 1: note = lipidated cysteine
CSSFNEKGEVSEKIITRADGTRLEYTGIKSDGSGKAKEVLKNFTLEGKVANDKTTLVVKCGTVTLSKNISKSGEVSVELNDTDSSAATKKTAAWNSGTSTLTITVNSKKTKDLVFTKENTITVQQYDSNGTKLEGSAVEITKLDEICNALKGTSDKNNGSGSKEKNKDGKYSFNEKGELSAKTMTRENGTKLEYTEMKSDGTGKAKEVLKNFTLEGKVANDKVTLEVKCGTVTLSKEIAKSGEVTVALNDTNTTQATKKTGAWDSKTSTLTISVNSKKTTQLVFTKQDTITVQKYDSAGTNLEGTAVEIKTLDELCNALK

SEQ ID NO: 2 - protein, 322 aa, synthetic construct
  LIPID 1: note = lipidated cysteine
CSSFNAKGELSEKTILRANGTRLEYTEIKSDGTGKAKEVLKDFALEGTLAADKTTLKVTCGTVVLSKHIPNSGEITVELNDSNSTQATKKTGKWDSNTSTLTISVNSKKTKNIVFTKEDTITVQKYDSAGTNLEGNAVEIKTLDELCNALKGTSDKNNGSGSKEKNKDGKYSFNEKGEVSEKILTRSNGTTLEYSQMTDAENATKAVETLKNGIKLPGNLVGGKTKLTVTCGTVTLSKNISKSGEITVALNDTETTPADKKTGEWKSDTSTLTISKNSQKTKQLVFTKENTITVQNYNRAGNALEGSPAEIKDLAELCAALK

SEQ ID NO: 3 - protein, 321 aa, synthetic construct
  LIPID 1: note = lipidated cysteine
CSSFNEKGEISEKTIVRANGTRLEYTDIKSDKTGKAKEVLKDFTLEGTLAADGKTTLKVTCGTVTLSKNISKSGEITVALDDTDSSGNKKSGTWDSGTSTLTISKNRTKTKQLVFTKEDTITVQNYDSAGTNLEGKAVEITTLKELCNALKGTSDKNNGSGSKEKNKDGKYSFNGKGETSEKTIVRANGTRLEYTDIKSDGSGKAKEVLKDFTLEGTLAADGKTTLKVTCGTVVLSKNILKSGEITAALDDSDTTRATKKTGKWDSKTSTLTISVNSQKTKNLVFTKEDTITVQRYDSAGTNLEGKAVEITTLKELCNALK

SEQ ID NO: 4 - DNA, 1020 bp, synthetic construct
  source 1..1020: mol_type = other DNA
atgaaagctactaaactggtactgggcgcggtaatcctgggttctactctgctggcaggttgctcaagcttcaacgaaaagggcgaagtcagcgaaaaaatcattacccgcgcagacggcacccgcctggaatacaccggcatcaaatcggacggcagcggcaaagcgaaagaagttctgaaaaactttaccctggaaggcaaagtcgcaaatgataaaaccaccctggtggtgaaatgcggcaccgttacgctgagcaaaaacattagtaaatccggtgaagtctctgtggaactgaatgataccgacagctctgcggccaccaagaaaaccgcagcttggaactcaggcacctcgacgctgaccattacggttaatagcaagaaaaccaaagatctggtcttcacgaaagaaaacaccatcacggtgcagcaatatgacagcaatggtaccaaactggaaggctccgctgtggaaatcacgaaactggatgaaatctgtaatgctctgaaaggtactagtgacaaaaacaatggctctggtagcaaagagaaaaacaaagatggcaagtactcattcaacgaaaaaggcgaactgtcggcgaaaacgatgacgcgtgaaaacggcaccaaactggaatatacggaaatgaaaagcgatggcaccggtaaagcgaaagaagttctgaaaaactttaccctggaaggcaaagtcgccaatgacaaagtcaccctggaagtgaaatgcggcaccgttacgctgtcaaaagaaattgcaaaatcgggtgaagtgaccgttgctctgaacgatacgaataccacgcaagcgaccaagaaaaccggcgcctgggacagcaaaacctctacgctgaccattagtgttaatagcaagaaaaccacgcagctggtcttcaccaaacaagatacgatcaccgtgcagaaatacgacagtgcgggtaccaacctggaaggcacggctgttgaaatcaaaaccctggacgaactgtgtaacgccctgaaa

SEQ ID NO: 5 - DNA, 1029 bp, synthetic construct
  source 1..1029: mol_type = other DNA
atgaaagctactaaactggtactgggcgcggtaatcctgggttctactctgctggcaggttgctcaagcttcaatgctaagggcgaactgagcgaaaaaacgatcctgcgtgcgaatggcacccgtctggaatacaccgaaatcaaatccgatggtacgggcaaagcaaaggaagtcctgaaagattttgctctggaaggtaccctggcggccgacaaaaccacgctgaaggtgacgtgcggcaccgtggttctgagcaaacatattccgaactctggtgaaatcaccgttgaactgaacgatagcaattctacgcaggcaaccaaaaagacgggcaaatgggacagtaatacctccacgctgaccatttcagtcaactcgaaaaagaccaaaaatattgtgttcacgaaggaagatacgatcaccgttcaaaaatatgactccgcgggcaccaacctggaaggcaatgccgtcgaaatcaaaaccctggatgaactgtgtaacgccctgaagggtactagtgacaaaaacaatggctctggtagcaaagagaaaaacaaagatggcaagtactcattcaacgaaaaaggcgaagtgagcgaaaaaattctgacccgtagcaatggcaccaccctggaatatagccagatgaccgatgcagaaaatgcaaccaaagcagttgaaaccctgaaaaacggtattaaactgcctggtaatctggttggtggtaaaaccaaactgaccgttacctgtggcaccgttaccctgagcaaaaacattagcaaaagcggtgaaattaccgtggcactgaatgataccgaaaccacaccggcagacaaaaaaaccggtgaatggaaaagcgataccagcaccctgaccattagtaaaaatagccagaaaacaaaacagctggtgtttaccaaagaaaacaccattaccgtgcagaattataaccgtgcaggtaatgcactggaaggtagtccggcagaaattaaagatctggcagaactgtgtgcagccctgaaataa

SEQ ID NO: 6 - DNA, 1023 bp, synthetic construct
  source 1..1023: mol_type = other DNA
atgaaagctactaaactggtactgggcgcggtaatcctgggttctactctgctggcaggttgctcaagcttcaacgaaaagggcgaaatctcagaaaaaaccatcgtccgcgctaacggcacccgcctggaatacaccgacatcaaatcagacaagaccggtaaagcgaaggaagttctgaaagattttacgctggaaggtaccctggcagcagacggtaaaaccacgctgaaggtgacctgcggtaccgttacgctgtccaaaaacattagtaagtccggcgaaatcacggtcgccctggatgacaccgatagctctggcaacaaaaagagcggtacctgggattcaggcacctcgacgctgaccatttctaaaaatcgtacgaaaaccaagcagctggtcttcacgaaagaagatacgatcaccgtgcaaaactatgacagcgcaggtaccaatctggaaggcaaagctgtggaaattaccacgctgaaagaactgtgtaatgctctgaaaggtactagtgacaaaaacaatggctctggtagcaaagagaaaaacaaagatggcaagtactcattcaacggcaaaggtgaaacgagcgaaaagaccatcgtgcgtgcgaacggtacccgcctggaatatacggacattaaatcggacggcagcggcaaagcaaaggaagtcctgaaagattttacgctggaaggtaccctggcagcagacggtaaaaccacgctgaaggtgacgtgcggcaccgtggttctgtcaaaaaacattctgaagtcgggtgaaatcaccgcagctctggatgacagcgataccacgcgtgctacgaaaaagaccggtaaatgggatagcaagacctctacgctgaccattagtgtcaactcccagaaaacgaagaatctggtgttcaccaaagaagatacgatcaccgttcaacgctatgacagtgcgggcaccaacctggaaggcaaagccgttgaaattaccacgctgaaagaactgtgtaatgctctgaaa

SEQ ID NO: 7 - protein, 21 aa, synthetic construct
GTSDKNNGSGSKEKNKDGKYS

SEQ ID NO: 8 - protein, 11 aa, synthetic construct
NFTLEGKVAND

(19) United States
(12) Patent Application Publication
(10) Pub. No.: US 2025/0281589 A1
(43) Pub. Date: Sep. 11, 2025

(54) COMPOSITIONS AND METHODS FOR ELICITING AN IMMUNE RESPONSE PROTECTIVE AGAINST LYME DISEASE

(71) Applicant: Pfizer Inc., New York, NY (US)

(72) Inventors: Annaliesa Sybil Anderson, Upper Saddle River, NJ (US); Steven Russell Bailey, Bryn Mawr, PA (US); Nicole Bézay, Hinterbrühl (AT); Susanne Eder-Lingelbach, Vienna (AT); William Carl Gruber, Warwick, NY (US); Romana Hochreiter, Wien (AT); Kathrin Ute Jansen, New York, NY (US); Thomas Lingelbach, Vienna (AT); Urban Lundberg, Pressbaum (AT); Daniel Alfred Scott, Spring City, PA (US); Raphael Simon, Upper Saddle River, NJ (US)

(73) Assignee: Pfizer Inc., New York, NY (US)

(21) Appl. No.: 18/859,577

(22) PCT Filed: Apr. 24, 2023
(86) PCT No.: PCT/US2023/066116

§ 371 Date: Oct. 24, 2024

Related U.S. Application Data
(60) Provisional application No. 63/334,271, filed on Apr. 25, 2022.
(60) Provisional application No. 63/357,398, filed on Jun. 30, 2022.
(60) Provisional application No. 63/429,483, filed on Dec. 1, 2022.

(51) Int. Cl. A61K 39/02; A61K 39/00; A61K 47/02; A61K 47/20; A61K 47/26; A61P 31/04

(52) U.S. Cl. CPC A61K 39/0225; A61K 47/02; A61K 47/20; A61K 47/26; A61P 31/04; A61K 2039/545; A61K 2039/55; A61K 2039/55505; A61K 2039/575

(57) ABSTRACT

The present invention relates to methods of administering a composition comprising the OspA fusion proteins of SEQ ID NO: 1 (LipSI D1-S2D1), SEQ ID NO: 2 (Lip-S4D1-S3hybD1), and SEQ ID NO: 3 (Lip-S5D1-S6D1) for eliciting an immune response protective against Lyme disease in a subject, such as vaccinating a subject against Lyme disease, and for treating, preventing, and/or reducing the risk of Lyme disease.

### REFERENCE TO AN ELECTRONIC SEQUENCE LISTING

The contents of the electronic sequence listing (1042270147WO00-SEQ-JRV.xml; Size: 13,099 bytes; and Date of Creation: Apr. 18, 2023) is herein incorporated by reference in its entirety.

### FIELD

The present invention is directed to methods of eliciting an immune response protective against Lyme disease in a subject and compositions for use thereof.

### BACKGROUND

Lyme disease is the most prevalent vector-borne disease in humans across the temperate regions of the northern hemisphere, with hundreds of thousands of people in North America (US and Canada) and Eurasia affected annually. In the US, the Northeast, mid-Atlantic, and upper Midwest regions are most affected, with an estimated 476,000 patients diagnosed and treated annually from 2010 to 2018. In Europe, the incidence, based on notified cases reported, is approximately 65,000 to 85,000 cases per year. However, because of inconsistent case reporting and the fact that Lyme disease is often undiagnosed, this number is largely underestimated. Lyme disease can occur at any age; however, incidence peaks in children 5 to 15 years of age and adults >50 years of age.

Lyme disease is caused by Borrelia burgdorferi sensu lato spirochetes, which are transmitted to humans via the bite of an Ixodes hard tick during a blood meal. The most common clinical manifestation of Lyme disease is a gradually expanding erythematous skin rash known as erythema migrans (EM). EM appears within days to weeks (average ˜1 to 2 weeks) at the location of a tick bite and is often accompanied by symptoms of fatigue, fever, headache, mild stiffness of the neck, arthralgia, or myalgia. Early borrelial infection can be missed because of objective signs and symptoms that are either not evident or not reported. If untreated or inadequately treated with antibiotics, the infection can disseminate via the bloodstream to other parts of the body, where it can cause serious manifestations affecting the nervous system, joints, or heart.

Lyme disease is both increasing in incidence and spreading geographically. More reliable preventive measures, such as a vaccine, are needed to further help reduce the risk of acquiring this potentially devastating disease. Currently, there is no licensed human Lyme disease vaccine available, and no other human Lyme disease vaccine candidate is in active clinical development. Thus there is a significant unmet medical need for a safe and efficacious Lyme disease vaccine.

### SUMMARY OF THE INVENTION

The present invention provides for methods of administering a composition comprising the OspA fusion proteins of SEQ ID NO: 1 (LipS1 D1-S2D1), SEQ ID NO: 2 (Lip-S4D1-S3hybD1), and SEQ ID NO: 3 (Lip-S5D1-S6D1) for eliciting an immune response protective against Lyme disease in a subject, such as vaccinating a subject against Lyme disease, and for treating, preventing, and/or reducing the risk of Lyme disease. In particular, administering at least three doses or at least two doses of the composition in adult and/or pediatric subjects to elicit a strong immunogenicity profile.

Accordingly, in some aspects, the present invention provides a method of eliciting an immune response protective against Lyme disease (Lyme borreliosis) in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose, wherein the method comprises administering at least three doses of the composition.

In some aspects, the present invention provides a method for treating, preventing or reducing the risk of Lyme disease (Lyme borreliosis) in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose, wherein the method comprises administering at least three doses of the composition.

In some aspects, the present invention provides a method of vaccinating a subject against Lyme disease (Lyme borreliosis), the method comprising administering to the subject a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose, wherein the method comprises administering at least three doses of the composition.

In some embodiments, a second dose is administered in a period of at least 6 weeks to at most 3 months after a first dose is administered, and a third dose is administered in a period of at least 5 months to at most 9 months after the first dose is administered. In some embodiments, a second dose is administered in a period of at least 6 weeks to at most 3 months after a first dose is administered, and a third dose is administered in a period of at least 5 months to at most 7 months after the first dose is administered. In some embodiments, a second dose is administered in a period of at least 50 days to at most 70 days after a first dose is administered, and a third dose is administered in a period of at least 5 months to at most 9 months after the first dose is administered. In some embodiments, a second dose is administered in a period of at least 50 days to at most 70 days after a first dose is administered, and a third dose is administered in a period of at least 170 days to at most 190 days after the first dose is administered. In some embodiments, a first dose is administered on day 1, a second dose is administered about 2 months after the first dose, and a third dose is administered about 5 months to about 9 months after the first dose is administered. In some embodiments, a first dose is administered on day 1, a second dose is administered about 2 months after the first dose, and a third dose is administered about 6 months after the first dose is administered.

In some embodiments, a fourth dose is administered in the period of at least 15 months to at most 21 months, particularly in the period of at least 17 months to at most 19 months after the first dose is administered. In some embodiments, a fourth dose is administered about 18 months after the first dose is administered. In some embodiments, a fourth dose is administered about 12 months after the third dose is administered.

In some embodiments, the total protein content of the three fusion proteins is 135 μg or 180 μg per dose, preferably 180 μg per dose, wherein a first dose is administered on day 1, a second dose is administered in a period of at least 50 days to at most 70 days after a first dose, and a third dose is administered in a period of at least 5 months to at most 9 months, or at least 5 months to at most 7 months, or at least 170 days to at most 190 days after the first dose, and wherein optionally a fourth dose is administered about 18 months after the first dose is administered. In some embodiments, a fourth dose is administered about 12 months after the third dose is administered.

In some embodiments, the subject is an adult subject, wherein the total protein content of the three fusion proteins is 135 μg or 180 μg per dose, preferably 180 μg per dose, wherein a first dose is administered on day 1, a second dose is administered in a period of at least 50 days to at most 70 days after a first dose, and a third dose is administered in a period of at least 5 months to at most 9 months, or at least 5 months to at most 7 months or at least 170 days to at most 190 days after the first dose, and wherein optionally a fourth dose is administered about 18 months after the first dose is administered or about 12 months after the third dose is administered.

In some embodiments, the subject is a pediatric subject, wherein the subject is a pediatric subject, wherein the total protein content of the three fusion proteins is 135 μg or 180 μg per dose, preferably 180 μg per dose, wherein a first dose is administered on day 1, a second dose is administered in a period of at least 50 days to at most 70 days after a first dose, and a third dose is administered in a period of at least 5 months to at most 9 months, or at least 5 months to at most 7 months or at least 170 days to at most 190 days after the first dose, and wherein optionally a fourth dose is administered about 18 months after the first dose is administered or about 12 months after the third dose is administered.

In some embodiments, the subject is a pediatric subject from birth to 4 years, wherein the total protein content of the three fusion proteins is 67.5 μg or 90 μg per dose, wherein a first dose is administered on day 1, a second dose is administered in a period of at least 50 days to at most 70 days after a first dose, and a third dose is administered in a period of at least 5 months to at most 9 months, or at least 5 months to at most 7 months or at least 170 days to at most 190 days after the first dose, and wherein optionally a fourth dose is administered about 18 months after the first dose is administered or about 12 months after the third dose is administered.

In some embodiments, a further dose is administered every year after the third dose or the optional fourth dose is administered, particularly after 1 year, after 2 years, and after 3 years.

In some aspects, the present invention provides a method of eliciting an immune response protective against Lyme disease in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins in the range of from 60 μg to 200 μg per dose, wherein the method comprises administering at least two doses of the composition.

In some aspects, the present invention provides a method for treating, preventing or reducing the risk of Lyme disease in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins in the range of from 60 μg to 200 μg per dose, wherein the method comprises administering at least two doses of the composition.

In some aspects, the present invention provides a method of vaccinating a subject against Lyme disease, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins in the range of from 60 μg to 200 μg per dose, wherein the method comprises administering at least two doses of the composition.

In some embodiments, a second dose is administered in a period of at least 5 months to at most 7 months after a first dose is administered. In some embodiments, a second dose is administered in a period of at least 170 days to at most 190 days after a first dose is administered. In some embodiments, a first dose is administered on day 1 and a second dose is administered about 6 months after the first dose is administered.

In some embodiments, a third dose is administered in the period of at least 15 months to at most 21 months, particularly in the period of at least 17 months to at most 19 months after the first dose is administered. In some embodiments, a third dose is administered about 18 months after the first dose is administered.

In some embodiments, the total protein content of the three fusion proteins is 135 μg or 180 μg per dose, preferably 180 μg per dose, wherein a first dose is administered on day 1, a second dose is administered in a period of at least 170 days to at most 190 days after the first dose, and wherein optionally a third dose is administered about 18 months after the first dose is administered.

In some embodiments, the subject is an adult subject, wherein the total protein content of the three fusion proteins is 135 μg or 180 μg per dose, preferably 180 μg per dose, wherein a first dose is administered on day 1, a second dose is administered in a period of at least 170 days to at most 190 days after the first dose, and wherein optionally a third dose is administered about 18 months after the first dose is administered.

In some embodiments, the subject is a pediatric subject, wherein the total protein content of the three fusion proteins is 135 μg or 180 μg per dose, preferably 180 μg per dose, wherein a first dose is administered on day 1, a second dose is administered in a period of at least 170 days to at most 190 days after the first dose, and wherein optionally a third dose is administered about 18 months after the first dose is administered.

In some embodiments, the subject is a pediatric subject from birth to 4 years, wherein the total protein content of the three fusion proteins is 67.5 μg or 90 μg per dose, wherein a first dose is administered on day 1, a second dose is administered in a period of at least 170 days to at most 190 days after the first dose, and wherein optionally a third dose is administered about 18 months after the first dose is administered.

In some embodiments, a further dose is administered every year after the second dose or the optional third dose is administered, particularly after 1 year, after 2 years, and after 3 years.

In some embodiments, the subject is a human subject. In some embodiments, the subject is aged 5 years or older. In some embodiments, the subject is aged 5-65 years. In some embodiments, the subject is an adult subject aged 18 years or older, such as 18-65 years and/or 50 years or older. In some embodiments, the subject a pediatric subject from birth to 17 years. In some embodiments, the subject is the pediatric subject is aged 5-17 years, such as 5-11 years (child) and/or 12-17 years (adolescent). In some embodiments, the pediatric subject is from birth to 4 years, such as 1-4 years and/or 2-4 years.

In some embodiments, the total protein content of the three fusion proteins is 135 μg or 180 μg per dose. In some embodiments, the total protein content of the three fusion proteins is 180 μg per dose. In some embodiments, the total protein content of the three fusion proteins is 67.5 μg or 90 μg per dose.

In some embodiments, the three fusion proteins comprise at least 60%, preferably at least 70%, more preferably at least 80% of all proteins in the composition. In some embodiments, the composition comprises the three fusion proteins in a weight ratio of 1:1:1 (Lip-S1 D1-S2D1:Lip-S4D1-S3hybD1:Lip-S5D1-S6D1).

In some embodiments, the composition elicits an immune response comprising an anti-OspA serotype 1, an anti-OspA serotype 2, an anti-OspA serotype 3, an anti-OspA serotype 4, ant-OspA serotype 5 and/or an anti-OspA serotype 6 antibody response. In some embodiments, the composition elicits an immune response comprising antibodies against Borrelia serotypes 1, 2, 3, 4, 5, and 6.

In some embodiments, the composition elicits an immune response comprising antibodies against Borrelia serotypes 1, 2, 3, 4, 5, and/or 6, wherein the immune response after administering at least three doses of the composition to an adult subject is higher than the immune response after administering at least two doses of the composition to an adult subject.

In some embodiments, geometric mean titers (GMTs) of antibodies against Borrelia serotypes after administering at least three doses of the composition to an adult subject are at least 2.0-fold higher than the GMTs after administering at least two doses of the composition to an adult subject. In some embodiments, the GMTs of antibodies against Borrelia serotypes after administering at least three doses of the composition to an adult subject are about 2.0-fold to about 3.0-fold-higher than the GMTs after administering at least two doses of the composition to an adult subject.

In some embodiments, the composition elicits an immune response comprising antibodies against Borrelia serotypes 1, 2, 3, 4, 5, and/or 6, wherein the immune response after administering a second dose of the composition to a pediatric subject is at least as high as or higher than the immune response after administering a third dose of the composition to an adult subject.

In some embodiments, the composition elicits an immune response comprising antibodies against Borrelia serotypes 1, 2, 3, 4, 5, and/or 6, wherein the immune response after administering at least three doses of the composition to a pediatric subject is higher than the immune response after administering at least three doses of the composition to an adult subject. In some embodiments, the GMTs of antibodies against Borrelia serotypes after administering at least three doses of the composition to a pediatric subject are at least 2.0-fold higher than the GMTs after administering at least three doses of the composition to an adult subject.

In some embodiments, the GMTs of antibodies against Borrelia serotypes after administering at least three doses of the composition to a pediatric subject aged 12-17 years are about 2.0-fold to about 3.0-fold higher that the GMTs after administering at least three doses of the composition to an adult subject. In some embodiments, the GMTs of antibodies against Borrelia serotypes after administering at least three doses of the composition to a pediatric subject aged 5-11 years are about 3.0-fold to about 5.0-fold higher that the GMTs after administering at least three doses of the composition to an adult subject.

In some embodiments, the composition elicits an immune response comprising antibodies against Borrelia serotypes 1, 2, 3, 4, 5, and/or 6, wherein the immune response after administering at least two doses of the composition to a pediatric subject is higher than the immune response after administering at least two doses of the composition to an adult subject. In some embodiments, GMTs of antibodies against Borrelia serotypes after administering at least two doses of the composition to a pediatric subject are at least 2.0-fold higher than the GMTs after administering at least two doses of the composition to an adult subject.

In some embodiments, the GMTs of antibodies against Borrelia serotypes after administering at least two doses of the composition to a pediatric subject aged 12-17 years are about 4.0-fold to about 6.0-fold higher that the GMTs after administering at least two doses of the composition to an adult subject. In some embodiments, the GMTs of antibodies against Borrelia serotypes after administering at least two doses of the composition to a pediatric subject aged 5-11 years are about 5.0-fold to about 8.0-fold higher than the GMTs after administering at least two doses of the composition to an adult subject.

In some embodiments, the composition elicits an immune response comprising antibodies against Borrelia serotypes 1, 2, 3, 4, 5, and 6 that is sustained for at least about 60 days, for at least about 180 days, for at least about 365 days, or for at least about 540 days.

In some embodiments, the immune response is sustained above baseline for at least about 180 days after administering at least two doses or at least three doses of the composition. In some embodiments, geometric mean fold rises (GMFRs) of antibodies against Borrelia serotypes at least 180 days after administering at least three doses of the composition to a pediatric subject aged 5-11 are at least 2.0-fold higher than the GMFRs at baseline. In some embodiments, the GMFRs of antibodies against Borrelia serotypes at least 180 days after administering at least three doses of the composition to a pediatric subject aged 5-11 are about 2.0-fold to 7.0-fold higher than the GMFRs at baseline.

In some embodiments, the composition is administered to a subject in a volume of 0.25 ml, 0.5 ml, or 1.0 ml. In a preferred embodiment, the composition is administered to a subject in a volume of 0.5 ml.

In some embodiments, the composition comprises at least one of sodium phosphate, sodium chloride, sucrose and polysorbate 20. In some embodiments, the sodium phosphate is present at a concentration between 5 mM and 50 mM, the sodium chloride is present at a concentration between 100 mM and 200 mM, the sucrose is present at a concentration between 2.5% and 10% and the polysorbate is present at a concentration between 0.01% and 0.1%. In some embodiments, the composition comprises an adjuvant. In some embodiments, the adjuvant comprises an aluminum adjuvant. In some embodiments, the composition comprises less than 1.25 ppb copper. In some embodiments, the copper is in the form of an ion, particularly as Cu⁺ or Cu²⁺. In some embodiments, the composition comprises L-methionine. In some embodiments, the L-methionine is present in a concentration of at least 10 mmol/l. In some embodiments, the concentration of L-methionine in mol/I is at least equivalent with a concentration of copper in the composition.

In some embodiments, the composition further comprises a reactive compound, wherein the reactive compound is selected from a redox active compound, a radical building compound, a stabilizing compound and a combination of any thereof, especially wherein the reactive compound is selected from formaldehyde, ethanol, chloroform, trichloroethylene, acetone, Triton-X-100, deoxycholate, diethylpyrocarbonate, sulphite, Na₂S₂O₅, beta-proprio-lacton, polysorbate such as Tween 20®, Tween 80®, O₂, phenol, pluronic type copolymers, and a combination of any thereof.

The present invention further provides for the compositions described herein for use in eliciting an immune response protective against Lyme disease in a subject. In another embodiment, the present invention provides for the compositions described herein for use in treating, preventing or reducing the risk of Lyme disease in a subject. In another embodiment, the present invention provides for the compositions described herein for use in vaccinating a subject against Lyme disease. In another embodiment, the present invention provides for the compositions described herein for use as a medicament. In another embodiment, the present invention provides for the compositions described herein for use as a vaccine.

### BRIEF DESCRIPTION OF THE DRAWINGS

FIG. 1: Subject enrollment process in Part A (Main Study Phase) for Phase 2 clinical trial for the multivalent Borrelia vaccine investigating a three-dose or two-dose primary immunization schedule that is performed in an age-descending, staggered manner for three age cohorts.

FIGS. 2A and 2B: FIG. 2A) shows a study design (Part A: Main Study Phase, Part B: Booster Phase) for of Phase 2 clinical trial for the multivalent Borrelia vaccine investigating three-dose or two-dose primary immunization schedule of the vaccine, and booster dose, in a study population aged 5 to 65 years, and FIG. 2B) shows an alternative Part B: Booster Phase.

FIG. 3: Geometric Mean Titers (GMTs) for OspA serotype-specific IgG antibodies (STs 1-6) determined by ELISA on Day 208/Month 7.

FIG. 4: Geometric Mean Titers (GMTs) for OspA serotype specific IgG antibodies (STs 1-6) determined by ELISA by age cohort on Day 208/Month 7.

FIG. 5: Geometric Mean Titers (GMTs) for OspA serotype specific IgG antibodies (STs 1-6) determined by ELISA over time, Group 1 (three doses, M 0-2-6).

FIGS. 6A-6F: Reverse cumulative distribution curve at Day 208/Month 7 for ELISA % of subjects vs FIG. 6A) serotype 1, FIG. 6B) serotype 2, FIG. 6C) serotype 3, FIG. 6D) serotype 4, FIG. 6E) serotype 5 and FIG. 6F) serotype 6, OspA-specific IgG titer by age cohort.

FIG. 7: Seroconversion Rates (SCRs) by age cohort on Day 208/Month 7.

FIG. 8: Geometric Mean Titers (GMTs) for OspA serotype specific IgG antibodies (STs 1-6) determined by ELISA over time in M 0-2-6 group.

FIG. 9: Geometric Mean Titers (GMTs) for OspA serotype specific IgG antibodies (STs 1-6) determined by ELISA over time in M 0-6 group.

FIG. 10: Reverse cumulative distribution curve at Day 365/Month 12 for ELISA % of subjects vs ST1 OspA-specific IgG titer by age. Group 1 (M 0-2-6), Group 2 (M 0-6), Group 3 (Placebo), Cohort 1 (18-65 years), Cohort 2 (12-17 years), Cohort 3 (5-11 years).

FIG. 11: Reverse cumulative distribution curve at for ELISA % of subjects vs ST1 OspA-specific IgG titer over time in M 0-2-6 group.

FIG. 12: Reverse cumulative distribution curve at for ELISA % of subjects vs ST1 OspA-specific IgG titer over time in M 0-6 group.

FIG. 13: Seroconversion Rates (SCRs) by age cohort on Day 365/Month 12.

### DETAILED DESCRIPTION OF THE INVENTION

The multivalent Lyme borreliosis vaccine of the present invention comprises the immunodominant C-terminal part of the six most common OspA serotypes present in Europe (ST1 to ST6) and the US (ST1) and is designed to induce anti-OspA antibodies to neutralize the Borrelia within the tick gut, blocking its transmission to the vertebrate host. OspA-based vaccines require high and sustained antibody titers because of their mechanism of action (outside of the immunized host body, against bacteria in the tick vector gut). The vaccine and administration schedule of the present invention allows generation of high antibody titers, and continued maintenance of high antibody titers, to enable sufficient immune response activity against bacteria in the tick gut, for use in treating, preventing, and/or reducing the risk of Lyme borreliosis.

The Phase 2 study described herein is the first clinical study with the vaccine of the present invention which enrolled a pediatric population (5-17 years). The study compares the immunogenicity and safety of the vaccine of the present invention after administration of two (at Months 0 and 6) or three (at Months 0, 2 and 6) primary series doses in groups aged 5-11 years, 12-17 years and 18-65 years. In pediatric participants (5-17 years old) that received the vaccine in either the two-dose schedule or three-dose schedule, the vaccine was found to be more immunogenic than in adults with both vaccination schedules tested. A strong immunogenicity profile was observed in study participants aged 5-17 years one month after the primary vaccination series. The safety and tolerability profile observed in the pediatric participants (5-17 years) was similar to the safety profile previously observed in adult participants.

The present invention provides for methods of eliciting an immune response protective against Lyme disease in a subject comprising administering a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1). The present invention further provides for methods for treating, preventing or reducing the risk of Lyme disease in a subject comprising administering a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1). The present invention also provides for methods for vaccinating a subject comprising administering a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1).

The methods may comprise administering at least three doses or at least two doses of the composition to a subject. In one embodiment, the methods comprise administering at least three doses of the composition to a subject. Alternatively, the methods comprise administering at least two doses of the composition to a subject. The subject may be aged 5 years or older. The subject may be an adult subject (aged 18 years or older, including aged 18-65 years and/or aged 50 years or older) or a pediatric subject (from birth to 17 years, including aged 5-17 years, such as aged 5-11 years and 12-17 years, and including from birth to 4 years, such as aged 1-4 years and aged 2-4 years).

The composition may comprise a total protein content of the three fusion proteins in the range of from 60 μg to 200 μg per dose. In an embodiment of the present invention, the composition comprises a total protein content of the three fusion proteins of 135 μg per dose. In an embodiment of the present invention, the composition comprises a total protein content of the three fusion proteins of 180 μg per dose. In an embodiment of the present invention, the composition comprises a total protein content of the three fusion proteins of 67.5 μg or 90 μg per dose.

The methods may comprise administering at least three doses of the composition to an adult and/or pediatric subject. In a preferred embodiment, the composition is administered in at east three doses to an adult subject. In an embodiment of the invention, the composition is administered in at least three doses to a pediatric subject. Accordingly, a first dose may be administered on day 1, a second dose may be administered in a period of at least 50 days to at most 70 days after a first dose, and a third dose may be administered in a period of at least 5 months to at most 9 months, or at least 5 months to at most 7 months or at least 170 days to at most 190 days after the first dose, and optionally a fourth dose may be administered about 18 months after the first dose is administered or about 12 months after the third dose is administered.

Alternatively, the methods may comprise administering at least two doses of the composition to an adult and/or pediatric subject. In an embodiment of the invention, the composition is administered in at least two doses to an adult subject. In an embodiment of the invention, the composition is administered in at two doses to a pediatric subject. Accordingly, a first dose may be administered on day 1, a second dose may be administered in a period of at least 170 days to at most 190 days after the first dose, and optionally a third dose may be administered about 18 months after the first dose is administered.

The methods may comprise a further dose may be administered every year, particularly after 1 year, after 2 years, and after 3 years.

In a preferred embodiment, the methods comprise administering a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1) to an adult subject, wherein the total protein content of the three fusion proteins is 180 μg per dose, and the methods comprise administering at least three doses of the composition, wherein a first dose is administered on day 1, a second dose is administered in a period of at least 170 days to at most 190 days after the first dose, and optionally a third dose is administered about 18 months after the first dose is administered.

In a preferred embodiment, the methods comprise administering a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1) to a pediatric subject, wherein the total protein content of the three fusion proteins is 180 μg per dose, and the methods comprise administering at least three doses of the composition, wherein a first dose is administered on day 1, a second dose is administered in a period of at least 170 days to at most 190 days after the first dose, and optionally a third dose is administered about 18 months after the first dose is administered.

Alternatively, a preferred embodiment provides for methods comprising administering a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) to an adult subject, wherein the total protein content of the three fusion proteins is 180 μg per dose, and the methods comprise administering at least two doses of the composition, wherein a first dose is administered on day 1, a second dose is administered in a period of at least 170 days to at most 190 days after the first dose, and optionally a third dose is administered about 18 months after the first dose is administered.

Alternatively, a preferred embodiment provides for methods comprising administering a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) to an pediatric subject, wherein the total protein content of the three fusion proteins is 180 μg per dose, and the methods comprise administering at least two doses of the composition, wherein a first dose is administered on day 1, a second dose is administered in a period of at least 170 days to at most 190 days after the first dose, and optionally a third dose is administered about 18 months after the first dose is administered.

In the study of the present invention, in adults, Geometric Mean Titers (GMTs) for all six serotypes were statistically significantly higher in the three dose group (M 0-2-6) compared to the two dose group (M 0-6) at Day 208/Month 7. The GMTs of antibodies against Borrelia serotypes after administering at least three doses of the composition to an adult subject are at least 2.0-fold higher and/or about 2.0-fold to about 3.0-fold-higher than the GMTs after administering at least two doses of the composition to an adult subject. See Tables 2 and 3.

In an embodiment, the GMTs of antibodies against Borrelia serotypes after administering at least three doses of the composition to an adult subject are about 2.5-fold higher for serotype 1, about 2.0-fold higher for serotype 2, about 2.1-fold higher for serotype 3, about 2.4-fold higher for serotype 4, about 2.7-fold higher for serotype 5, and/or about 2.7-fold higher for serotype 6 than the GMTs after administering at least two doses of the composition to an adult subject. See Tables 2 and 3.

Further, in adults, Geometric Mean Fold Rises (GMFRs) for all six serotypes were statistically significantly higher in the three dose group (M 0-2-6) schedule compared to the two dose group (M 0-6). The GMFRs of antibodies against Borrelia serotypes after administering at least three doses of the composition to an adult subject are at least 1.5-fold higher and/or about 1.5-fold to about 3.0-fold-higher than the GMTs after administering at least two doses of the composition to an adult subject. See Table 6.

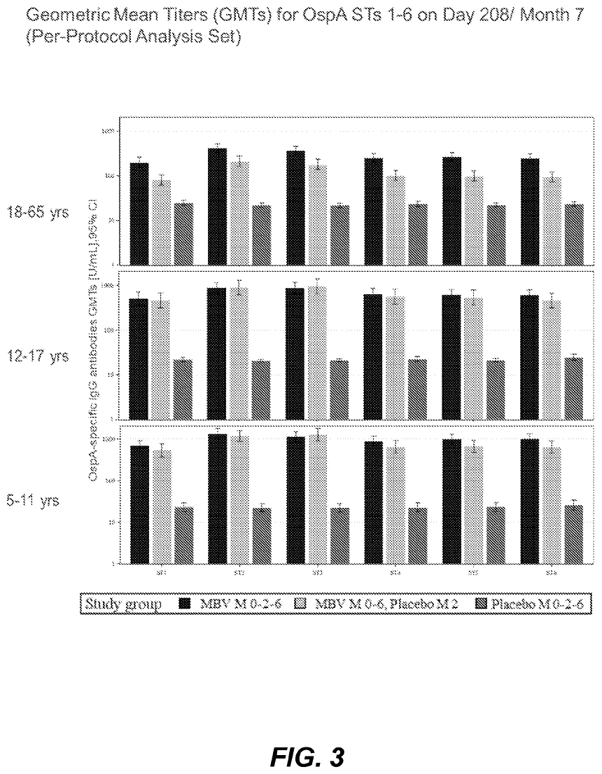

Thus, in an embodiment of the invention, the composition elicits an immune response comprising antibodies against Borrelia serotypes 1, 2, 3, 4, 5, and/or 6, wherein the immune response after administering three doses of the composition to an adult subject is higher than the immune response after administering two doses of the composition to an adult subject. See Example 1, FIG. 3.

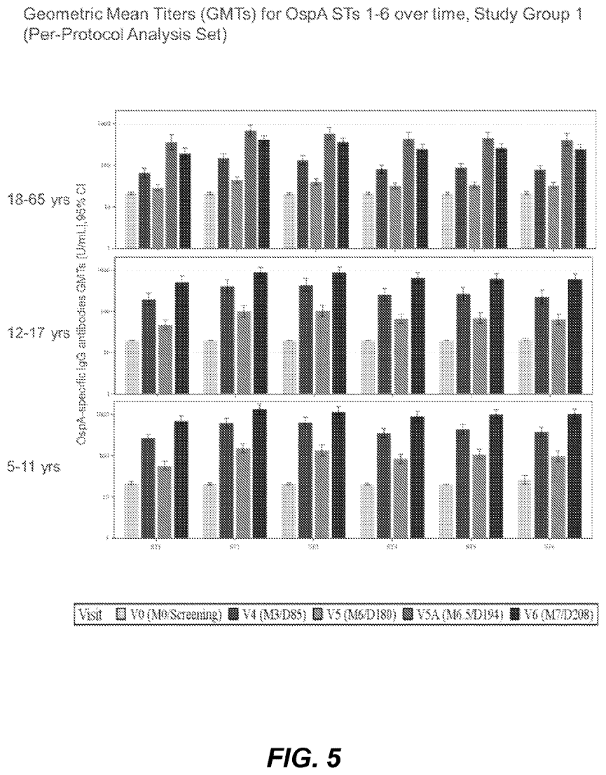

In the study of the present invention, in pediatric subjects (adolescents 12-17 years and children 5-11 years), already after a second dose of a M 0-2-6 schedule (i.e. at Day 85), the immune responses for all six serotypes were at least as high as after three doses (i.e. at Day 208) in adults 18-65 years. See Table 4. Thus, in an embodiment of the invention, the composition elicits an immune response comprising antibodies against Borrelia serotypes 1, 2, 3, 4, 5 and/or 6, wherein the immune response after administering a second dose of the composition to a pediatric subject is at least as high as or higher than the immune response after administering a third dose of the composition to an adult subject. See Example 1, FIG. 5.

In an embodiment of the invention, the composition elicits an immune response comprising antibodies against Borrelia serotypes 1, 2, 3, 4, 5, and/or 6, wherein the immune response after administering at least three doses of the composition to a pediatric subject is higher than the immune response after administering at least three doses of the composition to an adult subject. The GMTs of antibodies against Borrelia serotypes after administering at least three doses of the composition to a pediatric subject are at least 2.0-fold higher than the GMTs after administering at least three doses of the composition to an adult subject. See Table 2.

For pediatric subjects aged 12-17 years, the GMTs of antibodies against Borrelia serotypes after administering at least three doses of the composition to a pediatric subject aged 12-17 years are about 2.0-fold to about 3.0-fold higher that the GMTs after administering at least three doses of the composition to an adult subject. Further, the GMTs of antibodies against Borrelia serotypes after administering at least three doses of the composition to a pediatric subject aged 12-17 years are about 2.6-fold higher for serotype 1, about 2.2-fold higher for serotype 2, about 2.4-fold higher for serotype 3, about 2.6-fold higher for serotype 4, about 2.4-fold higher for serotype 5, and/or about 2.5-fold higher for serotype 6 than the GMTs after administering at least three doses of the composition to an adult subject.

For pediatric subjects aged 5-11 years, the GMTs of antibodies against Borrelia serotypes after administering at least three doses of the composition to a pediatric subject aged 5-11 years are about 3.0-fold to about 5.0-fold higher that the GMTs after administering at least three doses of the composition to an adult subject. Further, the GMTs of antibodies against Borrelia serotypes after administering at least three doses of the composition to a pediatric subject aged 5-11 years are about 3.5-fold higher for serotype 1, about 3.3-fold higher for serotype 2, about 3.1-fold higher for serotype 3, about 3.6-fold higher for serotype 4, about 3.6-fold higher for serotype 5, and/or about 4.1-fold higher for serotype 6 than the GMTs after administering at least three doses of the composition to an adult subject.

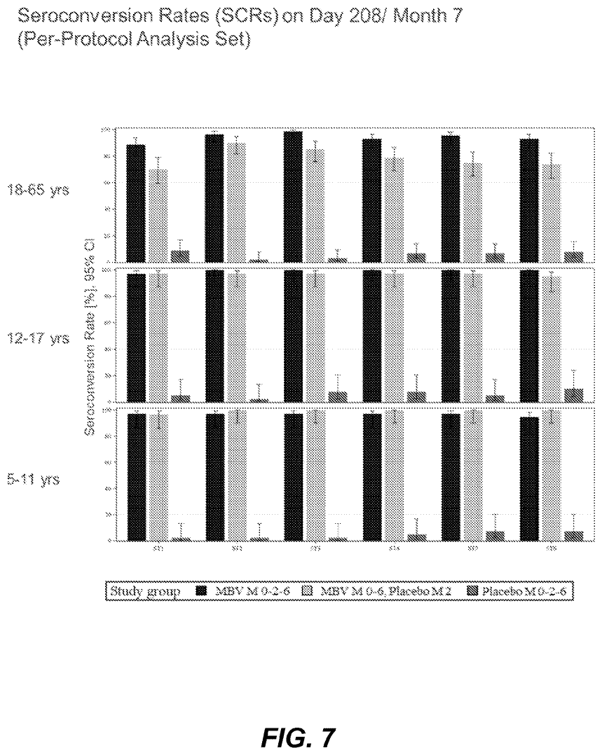

In an embodiment of the invention, the composition elicits an immune response comprising antibodies against Borrelia serotypes 1, 2, 3, 4, 5, and/or 6, wherein the immune response after administering at least two doses of the composition to a pediatric subject is higher than the immune response after administering at least two doses of the composition to an adult subject. See Example 1 and FIG. 7. The GMTs of antibodies against Borrelia serotypes after administering at least two doses of the composition to a pediatric subject are at least 2.0-fold higher than the GMTs after administering at least two doses of the composition to an adult subject. See Table 2.

For pediatric subjects aged 12-17 years, the GMTs of antibodies against Borrelia serotypes after administering at least two doses of the composition to a pediatric subject aged 12-17 years are about 4.0-fold to about 6.0-fold higher that the GMTs after administering at least two doses of the composition to an adult subject. Further, the GMTs of antibodies against Borrelia serotypes after administering at least two doses of the composition to a pediatric subject aged 12-17 years are about 5.8-fold higher for serotype 1, about 4.3-fold higher for serotype 2, about 5.4-fold higher for serotype 3, about 5.5-fold higher for serotype 4, about 5.5-fold higher for serotype 5, and/or about 4.9-fold higher for serotype 6 than the GMTs after administering at least two doses of the composition to an adult subject.

For pediatric subjects aged 5-11 years, the GMTs of antibodies against Borrelia serotypes after administering at least two doses of the composition to a pediatric subject aged 5-11 years are about 5.0-fold to about 8.0-fold higher than the GMTs after administering at least two doses of the composition to an adult subject. Further, the GMTs of antibodies against Borrelia serotypes after administering at least two doses of the composition to a pediatric subject aged 5-11 years are about 6.7-fold higher for serotype 1, about 5.7-fold higher for serotype 2, about 7.2-fold higher for serotype 3, about 6.3-fold higher for serotype 4, about 6.9-fold higher for serotype 5, and/or about 6.9-fold higher for serotype 6 than the GMTs after administering at least two doses of the composition to an adult subject.

In an embodiment of the invention, the composition elicits an immune response comprising antibodies against Borrelia serotypes 1, 2, 3, 4, 5, and 6 that is sustained for at least about 60 days, for at least about 180 days, for at least about 365 days, or for at least about 540 days. For example, the immune response is sustained above baseline for at least about 180 days after administering at least two doses or at least three doses of the composition. See Tables 9 and 10.

For pediatric subjects aged 5-11 years, the GMFRs of antibodies against Borrelia serotypes at least 180 days after administering at least three doses of the composition to a pediatric subject aged 5-11 are at least 2.0-fold higher and/or about 2.0-fold to 7.0-fold higher than the GMFRs at baseline. Further, GMFRs of antibodies against Borrelia serotypes at least about 180 days after administering at least three doses of the composition to a pediatric subject aged 5-11 years are about 2.8-fold higher for serotype 1, about 6.6-fold higher for serotype 2, about 5.4-fold higher for serotype 3, about 4.4-fold higher for serotype 4, about 5.5-fold higher for serotype 5, and/or about 4.3-fold higher for serotype 6 than the GMFRs at baseline. See Table 11.

In an embodiment of the present invention, the composition is administered in a volume of 0.25 ml, 0.5 ml, or 1.0 ml. In a preferred embodiment, the composition is administered in a volume of 0.5 mL.

### Lyme Disease

Lyme borreliosis (herein also referred to as “LB”), or Lyme disease (herein also referred to as “LD”), is the most commonly reported tick-borne disease in Europe and North America. The disease is caused by infection with the arthropod-borne gram-negative-like spirochete, Borrelia burgdorferi sensu lato (B. burgdorferi s.l.), and can involve multiple organs or tissues, resulting in skin, cardiac, musculoskeletal and neurological disorders.

In most countries, Lyme borreliosis is not a notifiable disease; therefore, exact data regarding annual incident rates are not available. In Europe, most human infections are caused by four Borrelia genospecies, presenting six OspA serotypes (STs): B. afzelii (ST2), B. garinii (ST3, ST5 and ST6), B. burgdorferi sensu stricto (s.s.) (ST1) and B. bavariensis (ST4). In the US, B. burgdorferi s.s. (ST1) is found in almost 100% of cases. Recently, a new genospecies named Borrelia mayonii has been described, which was found in few clinical specimen isolated in the Upper Midwest of the US [Pritt B S, et al. Identification of a novel pathogenic Borrelia species causing Lyme borreliosis with unusually high spirochaetaemia: a descriptive study, Lancet Infect Dis 2016; 16: 556-64].

Outer surface protein A (OspA) is one of the dominant proteins expressed by Borrelia burgdorferi s.l. when present in an unfed tick. During tick feeding the incoming blood signals the down regulation of OspA expression, allowing the spirochetes to penetrate the gut epithelium, migrate to the salivary glands and further into the blood of the host [Schwan T G and Piesman J. Temporal changes in outer surface proteins A and C of the Lyme disease-associated spirochete, Borrelia burgdorferi, during the chain of infection in ticks and mice. J Clin Microbiol 2000, 38:382-8]. OspA based LB vaccines induce antibodies that act on OspA expressing spirochetes within the tick gut, blocking transmission of the spirochete to the salivary glands and subsequently to the vertebrate host [de Silva A M and Telford S R 3rd, Brunet L R, Barthold S W, Fikrig E., J Exp Med. 1996, 183(1):271-5].

The multivalent Lyme borreliosis vaccine of the present invention comprises the immunodominant C-terminal part of the six most common OspA serotypes present in Europe (ST1 to ST6) and the US (ST1) and is designed to induce anti-OspA antibodies to neutralize the Borrelia within the tick gut, blocking its transmission to the host. The multimeric Borrelia vaccine has been shown to be highly protective against four Borrelia genospecies (B. burgdorferi s.s., B. afzelii, B. bavariensis and B. garinii), including five clinically relevant OspA serotypes (ST1, ST2, ST4 ST6) in mouse models using either infected ticks or in vitro grown spirochetes for challenge.

### Multimeric Borrelia Vaccine (“MBV”)

MBV is a multivalent Outer surface protein A (OspA) based vaccine designed for the prevention of Lyme borreliosis. The vaccine targets the majority of Borrelia strains expressing clinically relevant OspA serotypes (STs) present in the U.S. (ST1) and Europe (ST1 to ST6). The vaccine includes three proteins, each containing the C-terminal half of two OspA serotypes linked to form three fusion proteins of ˜35 kDa (ST1 and ST2, ST4 and ST3, and ST5 and ST6). MBV is used in a formulation that contains aluminum hydroxide adjuvant.

The compositions of the present invention comprise a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1), referred to as a multimeric Borrelia vaccine (“MBV”) herein.

The fusion proteins (sometimes also referred to as heterodimers) are derived from borrelial outer surface protein A (OspA). OspA is expressed by Borrelia only when it is in the gut of the tick vector. Thus, OspA antibodies produced by vaccination do not fight infection in the body, but enter the gut of the tick when it takes a blood meal. There, the antibodies likely neutralize the spirochetes and block the migration of bacteria from the midgut to the salivary glands of the tick, the route through which Borrelia enters the vertebrate host. Thus, OspA-specific antibodies prevent the transmission of Borrelia from the tick vector to the human host.

The above fusion proteins are lipidated mutant OspA fragment fusion proteins comprising a hybrid C-terminal OspA fragment, wherein the hybrid fragment consists of a C-terminal domain of OspA of Borrelia that is comprised of a fragment derived from OspA of a Borrelia strain different than B. garinii, strain PBr, and a second fragment of OspA from B. garinii, strain PBr, and differs from the corresponding wild-type sequence at least by the introduction of at least one disulfide bond. The disulfide bonds are disulfide bonds Type 1, wherein cysteine residues are inserted at position 183+/−3 and 270+/−3 (for further details see WO 2015/104396 A1, which is incorporated by reference herein in its entirety). S3hyb indicates a fusion of amino acids 125-176 of B. valaisiana and amino acids 177-274 of B. garinii, strain PBr. Lip means lipidation and indicates the N-terminal addition of glycerol and fatty acid residues. The “LN1” peptide linker is a fusion of two separate loop regions of the N-terminal half of OspA from B. burgdorferi s.s., strain B31 (aa 65-74 and aa 42-53, with an amino acid exchange at position 53 of D53S) which has the following sequence: GTSDKNNGSGSKEKNKDGKYS (SEQ ID NO: 7).

Particularly, Lip-S1 D1-S2D1 is a fusion protein of OspA serotype 1 and OspA serotype 2 with disulfide bond type 1 and comprises an N-terminal CSS for addition of lipids, a LN1 linker sequence and an N-terminal lipidation. Amino acids 164-174 of OspA serotype 1 are replaced by non-hLFA-1-like sequence NFTLEGKVAND (SEQ ID NO: 8). The sequence is shown as following SEQ ID NO: 1:

Lip-S1D1-S2D1-aa
SEQ ID NO: 1
LipCSSFNEKGEVSEKIITRADGTRLEYTGIKSDGSGKAKEVLKNFTLEGKVANDKTTLVVK
CGTVTLSKNISKSGEVSVELNDTDSSAATKKTAAWNSGTSTLTITVNSKKTKDLVFTKENTI
TVQQYDSNGTKLEGSAVEITKLDEICNALKGTSDKNNGSGSKEKNKDGKYSFNEKGELSAKT
MTRENGTKLEYTEMKSDGTGKAKEVLKNFTLEGKVANDKVTLEVKCGTVTLSKEIAKSGEVT
VALNDTNTTQATKKTGAWDSKTSTLTISVNSKKTTQLVFTKQDTITVQKYDSAGTNLEGTAV
EIKILDELCNALK

Lip-S4D1-S3hybD1 is a fusion protein of OspA serotype 4 and OspA serotype 3, comprising amino acids 125-176 of B. valaisiana, strain VS116 and amino acids 177-274 of B. garinii, strain PBr, serotype 3, with disulfide bond type 1 and comprises an N-terminal CSS for addition of lipids, an LN1 linker sequence and N-terminal lipidation. The sequence is shown as following SEQ ID NO: 2:

Lip-S4D1-S3hybD1-aa
SEQ ID NO: 2
LipCSSFNAKGELSEKTILRANGTRLEYTEIKSDGTGKAKEVLKDFALEGTLAADKTTLKVT
CGTVVLSKHIPNSGEITVELNDSNSTQATKKTGKWDSNISTLTISVNSKKTKNIVFTKEDTI
TVQKYDSAGTNLEGNAVEIKTLDELCNALKGTSDKNNGSGSKEKNKDGKYSFNEKGEVSEKI
LTRSNGTTLEYSQMTDAENATKAVETLKNGIKLPGNLVGGKTKLTVTCGTVTLSKNISKSGE
ITVALNDTETTPADKKTGEWKSDTSTLTISKNSQKTKQLVFTKENTITVQNYNRAGNALEGS
PAEIKDLAELCAALK

Lip-S5D1-S6D1 is a fusion protein of OspA serotypes 6 both with disulfide bond type 1, an N-terminal CSS for addition of lipids, LN1 linker sequence and an N-terminal lipidation. The sequence is shown as following SEQ ID NO: 3:

Lip-S5D1-S6D1-aa
SEQ ID NO: 3
LipCSSFNEKGEISEKTIVRANGTRLEYTDIKSDKTGKAKEVLKDFTLEGTLAADGKTTLKV
TCGTVTLSKNISKSGEITVALDDTDSSGNKKSGTWDSGTSTLTISKNRTKTKQLVFTKEDTI
TVQNYDSAGTNLEGKAVEITTLKELCNALKGTSDKNNGSGSKEKNKDGKYSFNGKGETSEKT
IVRANGTRLEYTDIKSDGSGKAKEVLKDFTLEGTLAADGKTTLKVTCGTVVLSKNILKSGEI
TAALDDSDTTRATKKTGKWDSKTSTLTISVNSQKTKNLVFTKEDTITVQRYDSAGTNLEGKA
VEITTLKELCNALK

The nucleic acid sequences coding for the above fusion proteins are as follows:

Lip-S1D1-S2D1-nt
SEQ ID NO: 4
ATGAAAGCTACTAAACTGGTACTGGGCGCGGTAATCCTGGGTTCTACTCTGCTGGCAGGTTG
CTCAAGCTTCAACGAAAAGGGCGAAGTCAGCGAAAAAATCATTACCCGCGCAGACGGCACCC
GCCTGGAATACACCGGCATCAAATCGGACGGCAGCGGCAAAGCGAAAGAAGTTCTGAAAAAC
TTTACCCTGGAAGGCAAAGTCGCAAATGATAAAACCACCCTGGTGGTGAAATGCGGCACCGT
TACGCTGAGCAAAAACATTAGTAAATCCGGTGAAGTCTCTGTGGAACTGAATGATACCGACA
GCTCTGCGGCCACCAAGAAAACCGCAGCTTGGAACTCAGGCACCTCGACGCTGACCATTACG
GTTAATAGCAAGAAAACCAAAGATCTGGTCTTCACGAAAGAAAACACCATCACGGTGCAGCA
ATATGACAGCAATGGTACCAAACTGGAAGGCTCCGCTGTGGAAATCACGAAACTGGATGAAA
TCTGTAATGCTCTGAAAGGTACTAGTGACAAAAACAATGGCTCTGGTAGCAAAGAGAAAAAC
AAAGATGGCAAGTACTCATTCAACGAAAAAGGCGAACTGTCGGCGAAAACGATGACGCGTGA
AAACGGCACCAAACTGGAATATACGGAAATGAAAAGCGATGGCACCGGTAAAGCGAAAGAAG
TTCTGAAAAACTTTACCCTGGAAGGCAAAGTCGCCAATGACAAAGTCACCCTGGAAGTGAAA
TGCGGCACCGTTACGCTGTCAAAAGAAATTGCAAAATCGGGTGAAGTGACCGTTGCTCTGAA
CGATACGAATACCACGCAAGCGACCAAGAAAACCGGCGCCTGGGACAGCAAAACCTCTACGC
TGACCATTAGTGTTAATAGCAAGAAAACCACGCAGCTGGTCTTCACCAAACAAGATACGATC
ACCGTGCAGAAATACGACAGTGCGGGTACCAACCTGGAAGGCACGGCTGTTGAAATCAAAAC
CCTGGACGAACTGTGTAACGCCCTGAAA
Lip-S4D1-S3hybD1-nt
SEQ ID NO: 5
ATGAAAGCTACTAAACTGGTACTGGGCGCGGTAATCCTGGGTTCTACTCTGCTGGCAGGTTG
CTCAAGCTTCAATGCTAAGGGCGAACTGAGCGAAAAAACGATCCTGCGTGCGAATGGCACCC
GTCTGGAATACACCGAAATCAAATCCGATGGTACGGGCAAAGCAAAGGAAGTCCTGAAAGAT
TTTGCTCTGGAAGGTACCCTGGCGGCCGACAAAACCACGCTGAAGGTGACGTGCGGCACCGT
GGTTCTGAGCAAACATATTCCGAACTCTGGTGAAATCACCGTTGAACTGAACGATAGCAATT
CTACGCAGGCAACCAAAAAGACGGGCAAATGGGACAGTAATACCTCCACGCTGACCATTTCA
GTCAACTCGAAAAAGACCAAAAATATTGTGTTCACGAAGGAAGATACGATCACCGTTCAAAA
ATATGACTCCGCGGGCACCAACCTGGAAGGCAATGCCGTCGAAATCAAAACCCTGGATGAAC
TGTGTAACGCCCTGAAGGGTACTAGTGACAAAAACAATGGCTCTGGTAGCAAAGAGAAAAAC
AAAGATGGCAAGTACTCATTCAACGAAAAAGGCGAAGTGAGCGAAAAAATTCTGACCCGTAG
CAATGGCACCACCCTGGAATATAGCCAGATGACCGATGCAGAAAATGCAACCAAAGCAGTTG
AAACCCTGAAAAACGGTATTAAACTGCCTGGTAATCTGGTTGGTGGTAAAACCAAACTGACC
GTTACCTGTGGCACCGTTACCCTGAGCAAAAACATTAGCAAAAGCGGTGAAATTACCGTGGC
ACTGAATGATACCGAAACCACACCGGCAGACAAAAAAACCGGTGAATGGAAAAGCGATACCA
GCACCCTGACCATTAGTAAAAATAGCCAGAAAACAAAACAGCTGGTGTTTACCAAAGAAAAC
ACCATTACCGTGCAGAATTATAACCGTGCAGGTAATGCACTGGAAGGTAGTCCGGCAGAAAT
TAAAGATCTGGCAGAACTGTGTGCAGCCCTGAAATAA
Lip-S5D1-S6D1-nt
SEQ ID NO: 6
ATGAAAGCTACTAAACTGGTACTGGGCGCGGTAATCCTGGGTTCTACTCTGCTGGCAGGTTG
CTCAAGCTTCAACGAAAAGGGCGAAATCTCAGAAAAAACCATCGTCCGCGCTAACGGCACCC
GCCTGGAATACACCGACATCAAATCAGACAAGACCGGTAAAGCGAAGGAAGTTCTGAAAGAT
TTTACGCTGGAAGGTACCCTGGCAGCAGACGGTAAAACCACGCTGAAGGTGACCTGCGGTAC
CGTTACGCTGTCCAAAAACATTAGTAAGTCCGGCGAAATCACGGTCGCCCTGGATGACACCG
ATAGCTCTGGCAACAAAAAGAGCGGTACCTGGGATTCAGGCACCTCGACGCTGACCATTTCT
AAAAATCGTACGAAAACCAAGCAGCTGGTCTTCACGAAAGAAGATACGATCACCGTGCAAAA
CTATGACAGCGCAGGTACCAATCTGGAAGGCAAAGCTGTGGAAATTACCACGCTGAAAGAAC
TGTGTAATGCTCTGAAAGGTACTAGTGACAAAAACAATGGCTCTGGTAGCAAAGAGAAAAAC
AAAGATGGCAAGTACTCATTCAACGGCAAAGGTGAAACGAGCGAAAAGACCATCGTGCGTGC
GAACGGTACCCGCCTGGAATATACGGACATTAAATCGGACGGCAGCGGCAAAGCAAAGGAAG
TCCTGAAAGATTTTACGCTGGAAGGTACCCTGGCAGCAGACGGTAAAACCACGCTGAAGGTG
ACGTGCGGCACCGTGGTTCTGTCAAAAAACATTCTGAAGTCGGGTGAAATCACCGCAGCTCT
GGATGACAGCGATACCACGCGTGCTACGAAAAAGACCGGTAAATGGGATAGCAAGACCTCTA
CGCTGACCATTAGTGTCAACTCCCAGAAAACGAAGAATCTGGTGTTCACCAAAGAAGATACG
ATCACCGTTCAACGCTATGACAGTGCGGGCACCAACCTGGAAGGCAAAGCCGTTGAAATTAC
CACGCTGAAAGAACTGTGTAATGCTCTGAAA

Further information on the fusion proteins and their production is derivable from WO 2015/104396 A1, which is incorporated by reference herein in its entirety, wherein Lip-S1 D1-S2D1, Lip-S4D1-S3hybD1 and Lip-S5D1-S6D1 correspond to SEQ ID NOs: 29, 27 and 33, respectively.

As detailed above, the fusion proteins are lipidated proteins, wherein the lipid moieties, along with the glycerol group, is also referred to as “Lip”. According to the invention, Lip comprises one to three lipids such as C₁₄₋₂₀ alkyl and/or C₁₄₋₂₀ alkenyl attached to a glycerol and an amino group of the N-terminal cysteine of the polypeptide of the invention, or preferably wherein Lip is a moiety of formula (I) below,

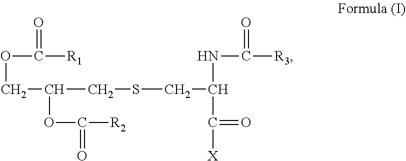

in which one of R₁, R₂ or R₃ is C₁₄-C₂₀ alkyl or alkenyl, and each of the others, independently is C₁₄-C₂₀ alkyl or C₁₄-C₂₀ alkenyl, and X is an amino acid sequence attached to the cysteine residue shown in Formula (I). More preferably, Lip plus the N-terminal cysteine of the polypeptide is N-palmitoyl-S-(2RS)-2,3-bis-(palmitoyloxy) propyl cysteine (referred to herein as “Pam₃Cys”) and is connected via the carbonyl C of the cysteine to said amino acid sequence of the invention. In Formula (I) above R₁, R₂ and R₃ would be palmitoyl moieties and X is an amino acid sequence attached to the cysteine residue.

The fusion proteins are encompassed in a composition. The composition is pharmaceutically acceptable, which allows for administration to a human. It may optionally contain any pharmaceutically acceptable carrier or excipient, such as buffer substances, stabilizers or further active ingredients, especially ingredients known in connection with pharmaceutical compositions and/or vaccine production. The composition may comprise sodium phosphate, sodium chloride, L-methionine, sucrose and Polysorbate-20 (Tween-20) at a pH of 6.7+/−0.2. Preferably, the pharmaceutical composition also comprises aluminium hydroxide, preferably at a concentration of 0.15%. Also the composition may comprise between 5 mM and 50 mM sodium phosphate, between 100 and 200 mM sodium chloride, between 5 mM and 25 mM L-methionine, between 2.5% and 10% (w/v) Sucrose, between 0.01% and 0.1% (v/v) Tween 20 and between 0.1% and 0.2% (w/v) aluminium hydroxide. More preferably, the formulation comprises 10 mM sodium phosphate, 150 mM sodium chloride, 10 mM L-methionine, 5% Sucrose, 0.05% Tween 20 and 0.15% (w/v) aluminium hydroxide at pH 6.7±0.2. In a preferred embodiment, the excipient is L-methionine.

In some embodiments, the composition comprising the fusion proteins are administered to a subject in a volume of about 0.25 milliliter (ml) to about 1.0 ml or greater, such as 0.25 ml, 0.3 ml, 0.4 ml, 0.5 ml, 0.6 ml, 0.7 ml, 0.8 ml, 0.9 ml, or 1.0 ml. In one embodiment, the volume administered to a subject is in the range of 0.25 ml to 1.0 ml, or 0.25 ml to 0.5 ml, or 0.5 ml to 1.0 ml. In one embodiment, the volume administered to a subject is about 0.25 ml, about 0.5 ml or about 1.0 ml. In one embodiment, the volume administered to a subject is 0.25 ml, 0.5 ml, or 1.0 ml. In one embodiment, the volume administered to a subject is 0.25 ml. In a preferred embodiment, the volume administered to a subject is 0.5 ml. In one embodiment, the volume administered to a subject is 1.0 ml. In a preferred embodiment, the volume administered to a subject who is 5 years or older (such as 5-17 years old, 5-11 years old, 12-17 years old, 18 years or older, 18-65 years old, or 50 years or older) is 0.5 ml or 1.0 ml. In a preferred embodiment, the volume administered to a subject who is 5 years or older (such as 5-17 years old, 5-11 years old, 12-17 years old, 18 years or older, 18-65 years old, or 50 years or older) is 1.0 ml. In a further preferred embodiment, the volume administered to a subject who is 5 years or older (such as 5-17 years old, 5-11 years old, 12-17 years old, 18 years or older, 18-65 years old, or 50 years or older) is 0.5 ml. In a further preferred embodiment, the volume administered to a subject who is 0-4 years old (such as 1-4 years old, or 2-4 years old) is 0.25 ml.

According to the invention, the composition is used as a vaccine, particularly against an infection caused by Borrelia species, more preferably pathogenic Borrelia species as disclosed herein more preferably comprising B. burgdorferi s.s., B. afzelii, B. bavariensis and B. garinii, and/or other pathogens against which the antigens have been included in the vaccine. Preferably, the Borrelia species is selected from B. burgdorferi s.s., B. garinii, B. afzelli, B. andersoni, B. bavariensis, B. bissettii, B. valaisiana, B. lusitaniae, B. spielmanii, B. japonica, B. tanukii, B. turdi or B. sinica infection, preferably a B. burgdorferi s.s., B. afzelii and B. garinii.

The composition for use according to the present invention may be administered to a human as an injectable composition, for example as a sterile aqueous dispersion, preferably isotonic. The composition may be administered via a systemic or mucosal route. These administrations may include injection via the intramuscular, intraperitoneal, intradermal or subcutaneous routes; or via mucosal administration to the oral/alimentary, respiratory or genitourinary tracts. Although the vaccine used in methods of the invention may be administered as a combination of the three fusion proteins, components thereof (e.g. the individual fusion proteins) may also be administered separately at the same time or sequentially or just as a one component vaccine (i.e. just Lip-S1D1-S2D1).

### Methods

In view of the special mode of action of this vaccine, which happens outside the human body, a need of constantly relatively high antibody titers throughout the tick seasons are of utmost importance. In addition, minimal natural boosting effect for present OspA-specific antibodies upon natural infection can be expected because OspA is only present on the surface of Borrelia as long as Borrelia bacteria are in the midgut of the tick, and not after antibodies have possibly entered the human host. Therefore, high antibody titers are needed and the composition is to be administered at least three times, or alternatively two times. In accordance with this, the composition may be administered two times, three times, four times, five times, or even more often.

It is advantageous to determine an administration schedule, e.g. number of doses and dose range, that provides a sustained protective effect against all Borrelia OspA serotypes for adult and pediatric subjects. In the present invention, a Phase 2 study is conducted in two parts: Main Study Phase (Part A) and Booster Phase (Part B). The study compares the safety and immunogenicity of two different primary immunization schedules applying at least three (Month 0-2-6) or at least two (Month 0-6) vaccinations. Within the study, 600 healthy subjects aged 5-65 years are included. Subjects with a history of Lyme borreliosis (previous infection with Borrelia) as well as Borrelia naïve subjects are enrolled. Adult subjects may be aged 18 years or older, such as 18-65 years and/or 50 years or older. Pediatric subjects may be from birth to age 17 years, including aged 5-17 years, such as 5-11 years (children) and/or 12-17 years (adolescents), and including younger children from birth to 4 years, such as 0-4 years, 1-4 years and/or 2-4 years.

In accordance with this administration schedule, the vaccine may be administered to a subject at least three times at a total protein content of the three fusion proteins in the range of from 60 μg to 200 μg per dose, or more preferably from 120 μg to 200 μg per dose. In some embodiments, the vaccine may be administered to a subject at least three times at a total protein content of the three fusion proteins at 135 μg or 180 μg per dose, or more preferably 180 μg per dose.

Alternatively, in accordance with this administration schedule, the vaccine may be administered to a subject at least two times at a total protein content of the three fusion proteins in the range of from 60 μg to 200 μg per dose, or more preferably from 120 to 200 μg per dose. In some embodiments, the vaccine may be administered to a subject at least times at a total protein content of the three fusion proteins at 135 μg or 180 μg per dose, or more preferably 180 μg per dose.

In accordance with this administration schedule, the vaccine may be administered to an adult subject at least three times at a total protein content of the three fusion proteins in the range of from 60 μg to 200 μg per dose, or more preferably from 120 μg to 200 μg per dose. In some embodiments, the vaccine may be administered to an adult subject at least three times at a total protein content of the three fusion proteins at 135 μg or 180 μg per dose, or more preferably 180 μg per dose.

Further, in accordance with this administration schedule, the vaccine may be administered to a pediatric subject at least three times at a total protein content of the three fusion proteins in the range of from 60 μg to 200 μg per dose, or from 120 μg to 200 μg per dose, or from 60 μg to 100 μg per dose. In some embodiments, the vaccine may be administered to a pediatric subject at least three times at a total protein content of the three fusion proteins at 135 μg or 180 μg per dose, or more preferably 180 μg per dose. In some embodiments, the vaccine may be administered to a pediatric subject at least three times at a total protein content of the three fusion proteins at 67.5 μg or 90 μg per dose.

Alternatively, in accordance with this administration schedule, the vaccine may be administered to an adult subject at least two times at a total protein content of the three fusion proteins in the range of from 60 μg to 200 μg per dose, or more preferably from 120 to 200 μg per dose. In some embodiments, the vaccine may be administered to an adult subject at least two times at a total protein content of the three fusion proteins at 135 μg or 180 μg per dose, or more preferably 180 μg per dose.

Further, alternatively, in accordance with this administration schedule, the vaccine may be administered to a pediatric subject at least two times at a total protein content of the three fusion proteins in the range of from 60 μg to 200 μg per dose, or from 120 μg to 200 μg per dose, or from 60 to 100 μg per dose. In some embodiments, the vaccine may be administered to a pediatric subject at least two times at a total protein content of the three fusion proteins at 135 μg or 180 μg per dose, or more preferably 180 μg per dose. In some embodiments, the vaccine may be administered to a pediatric subject at least two times at a total protein content of the three fusion proteins at 67.5 μg or 90 μg per dose.

For pediatric subjects, typically pediatric subjects from birth to 4 years (e.g. 0-4 years old, 1-4 years old, and/or 2-4 years old), half of the above doses may be applied, e.g. at 60 to 100, or at 67.5 μg or 90 μg (half dose), but the adult dose may also be appropriate e.g. at 120 μg to 200 μg (adult dose). Pediatric subjects aged 5 to 17 years, including children (age 5-11) and adolescents (age 12-17), may receive the adult dose.

The vaccines of the present invention, such as MBV, may be applied according to different primary immunization schedules. This includes a primary immunization schedule that applies at least three doses of the vaccine, such as administration at Month 0-2-6, and a primary immunization schedule that applies at least two doses of the vaccine, such as administration at Month 0-6. Further administrations may be booster doses for re-exposure to the vaccine and are applied typically every 12 months after the last dose of the primary immunization schedule, e.g. a first booster may be administered at month 18. Booster doses increase immunity back to protective levels, after titers have declined through time (see PCT Publication No. WO 2021/207615). It has been shown that boosters are useful for sustaining high OspA antibody levels which are needed to confer protection.

Therefore, the methods and compositions for use according to the present invention described herein are characterized in that, for a schedule that applies at least three doses, a second administration is in the period of at least 4 to 6 weeks to at most 3 months after the first administration and/or a third administration is in the period of at least 5 months to at most 9 months after the first administration. In accordance with this, the first administration occurs at time point zero (day 1), the second administration in the period of at least 4 weeks to at most 3 months thereafter and the third administration in the period of at least 5 months to at most 9 months after the first administration. Further a booster may be every year, e.g. about 12 months, 24 months, 36 months etc. after the third administration (i.e. after the month 6 immunization).

The methods and compositions for use according to the present invention described herein are further characterized in that, for a schedule that applies at least three doses, a second administration is in the period of at least 4 to 6 weeks to at most 3 months after the first administration and/or a third administration is in the period of at least 5 months to at most 7 months after the first administration. In accordance with this, the first administration occurs at time point zero (day 1), the second administration in the period of at least 4 weeks to at most 3 months thereafter and the third administration in the period of at least 5 months to at most 7 months after the first administration. Further a booster may be every year, e.g. about 12 months, 24 months, 36 months etc. after the third administration (i.e. after the month 6 immunization).

More preferably, the methods and compositions for use according to the present invention are characterized in that the second administration is in the period of at least 40 days to at most 80 days, particularly in the period of at least 50 days to at most 70 days, more particularly in the period of at least 50 days to at most 60 days, especially 56 or 57 days after the first administration.

Also more preferably, the methods and compositions for use according to the present invention are characterized in that the third administration is in the period of at least 5 months to at most 9 months, or at least 5 months to at most 7 months or, at least 120 days to at most 240 days, in the period of at least 170 days to at most 190 days, especially 180 days after the first administration.

In some cases, one or more further administrations may be needed or desirable. In accordance with this a fourth administration of the composition may be in the period of at least 15 months to at most 21 months, particularly in the period of at least 17 months to at most 19 months, especially about 18 months, after the first administration. In accordance with this a fourth administration of the composition may be in the period of about 12 months after the third administration.

In some embodiments, the methods and compositions for use according to the present invention are characterized in that the second administration is about 2 months (about 56 days) after the first administration, wherein the third administration is about 6 months (about 180 days) after the first administration, and wherein the optional fourth administration (first booster dose) is about 18 months after the first administration.

Alternatively, the methods and compositions for use according to the present invention described herein are characterized in that, for a schedule that applies at least two doses, a second administration is in the period of at least 5 months to at most 7 months after a first administration. In accordance with this, the first administration occurs at time point zero (day 1), the second administration in the period of at least 5 months to at most 7 months after the first administration. Further a booster may be every year, e.g. about 12 months, 24 months, 36 months etc. after the second administration (i.e. after the month 6 immunization).

Alternatively, more preferably, the methods and compositions for use according to the present invention are characterized in that the second administration is in the period of at least 120 days to at most 240 days, in the period of at least 170 days to at most 190 days, especially 180 days after the first administration.

In some cases, one or more further administrations may be needed or desirable. In accordance with this a third administration of the composition may be in the period of at least 15 months to at most 21 months, particularly in the period of at least 17 months to at most 19 months, especially about 18 months, after the first administration.

In some embodiments, the methods and compositions for use according to the present invention are characterized in the second administration is about 6 months (about 180 days) after the first administration, and wherein the optional third administration (first booster dose) is about 18 months after the first administration.

While booster doses typically are applied every 12 months, booster doses can be administered within several months of the 12 month timing, such as between 9-15 months after the final dose of the primary immunization (e.g., at 9 months, 10 months, 11 months, 12 months, 13 months, 14 months, or 15 months after primary immunization), or subsequent booster doses (e.g., at 9 months, 10 months, 11 months, 12 months, 13 months, 14 months, or 15 months after the first or subsequent booster doses). The booster doses may in general be administered prior to the tick season, e.g. around February to June in the Northern hemisphere and around September to December in the Southern hemisphere.

The present invention provides for methods for eliciting an immune response protective against Lyme disease (Lyme borreliosis) in a human subject, the methods comprise administering to a human subject a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1), at least three times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least three times in the range of from 60 to 100 μg per dose, more preferably 67.5 μg or 90 μg per dose, and most preferably 90 μg per dose (but a full dose may also be suitable).

Alternatively, the present invention provides for methods for eliciting an immune response protective against Lyme disease (Lyme borreliosis) in a human against, the methods comprise administering to a human subject a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1), at least two times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least two times in the range of from 60 to 100 μg per dose, more preferably 67.5 μg or 90 μg per dose, and most preferably 90 μg per dose (but a full dose may also be suitable).

In some embodiments, the methods comprise eliciting an immune response in a human subject protective against Lyme disease caused by Borrelia species, more preferably pathogenic Borrelia species as disclosed herein more preferably comprising B. burgdorferi s.s., B. afzelii, B. bavariensis, and B. garinii, and/or other pathogens against which the antigens have been included in the composition. Preferably, the composition is for use in a method of eliciting an immune response protective against B. burgdorferi s.s., B. garinii, B. afzelli, B. andersoni, B. bavariensis, B. bissettii, B. valaisiana, B. lusitaniae, B. spielmanii, B. japonica, B. tanukii, B. turdi, or B. sinica infection, preferably against B. burgdorferi s.s., B. afzelii and/or B. garinii.

The present invention provides for methods for treating, preventing or reducing the risk of Lyme disease (Lyme borreliosis) in a human subject, the methods comprise administering to a human subject a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1), at least three times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least three times in the range of from 60 to 100 μg per dose, more preferably 67.5 μg or 90 μg per dose, and most preferably 90 μg per dose (but a full dose may also be suitable).

Alternatively, the present invention provides for methods for treating, preventing or reducing the risk of Lyme borreliosis (Lyme disease) in a human against, the methods comprise administering to a human subject a composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1), at least two times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least two times in the range of from 60 to 100 μg per dose, more preferably 67.5 μg or 90 μg per dose, and most preferably 90 μg per dose (but a full dose may also be suitable).

The present invention provides for methods of vaccinating against Lyme borreliosis (Lyme disease) in a human subject, the methods comprise administering to a human subject a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1), at least three times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least three times in the range of from 60 to 100 μg per dose, more preferably 67.5 μg or 90 μg per dose, and most preferably 90 μg per dose (but a full dose may also be suitable).

Alternatively, the present invention provides for methods of vaccinating against Lyme borreliosis (Lyme disease) in a human against, the methods comprise administering to a human subject a composition comprising a fusion protein of SEQ ID NO: 1 (Lip-S1D1-S2D1), a fusion protein of SEQ ID NO: 2 (Lip-S4D1-S3hybD1) and a fusion protein of SEQ ID NO: 3 (Lip-S5D1-S6D1), at least two times at a total protein content of the three fusion proteins in the range of from 120 to 200 μg per dose, more preferably 135 μg or 180 μg per dose, and most preferably 180 μg per dose for a human adult subject or pediatric subject. For a pediatric subject (in particular younger children from birth to 4 years old, such as 1-4 years old, or 2-4 years old) a half dose may be applied at least two times in the range of from 60 to 100 μg per dose, more preferably 67.5 μg or 90 μg per dose, and most preferably 90 μg per dose (but a full dose may also be suitable).

In one embodiment, the compositions described herein and methods of use thereof according to the present invention are characterized in that the total protein content of the three fusion proteins is 135 μg per dose for a human subject, particularly an adult subject, wherein the second administration is about 2 months (about 56 days) after the first administration, wherein the third administration is about 5 months to about 9 months or about 6 months (about 180 days) after the first administration, and wherein the optional fourth administration (first booster dose) is about 18 months after the first administration or about 12 months after the third administration.

In one embodiment, the compositions described herein and methods of use thereof according to the present invention are characterized in that the total protein content of the three fusion proteins is 135 μg per dose for a human subject, particularly a pediatric subject, or may be only 67.5 μg per dose for a younger pediatric subject (in particular, a pediatric subject from birth to 4 years, such as 1-4 years old, or 2-4 years old), wherein the second administration is about 2 months (about 56 days) after the first administration, wherein the third administration is about 5 months to about 9 months or about 6 months (about 180 days) after the first administration, and wherein the optional fourth administration (first booster dose) is about 18 months after the first administration or about 12 months after the third administration.

In a preferred embodiment, the compositions described herein and methods of use thereof according to the present invention are characterized in that the total protein content of the three fusion proteins is 180 μg per dose for a human subject, particularly an adult subject, wherein the second administration is about 2 months (about 56 days) after the first administration, wherein the third administration is about 5 months to about 9 months or about 6 months (about 180 days) after the first administration, and wherein the optional fourth administration (first booster dose) is about 18 months after the first administration or about 12 months after the third administration.

In a preferred embodiment, the compositions described herein and methods of use thereof according to the present invention are characterized in that the total protein content of the three fusion proteins is 180 μg per dose for a human subject, particularly a pediatric subject, or may be only 90 μg per dose for a younger pediatric subject (in particular, a pediatric subject from birth to 4 years, such as 1-4 years old, or 2-4 years old), wherein the second administration is about 2 months (about 56 days) after the first administration, wherein the third administration is about 5 months to about 9 months or about 6 months (about 180 days) after the first administration, and wherein the optional fourth administration (first booster dose) is about 18 months after the first administration or about 12 months after the third administration.

Alternatively, in one embodiment, the compositions described herein and methods of use thereof according to the present invention are characterized in that the total protein content of the three fusion proteins is 135 μg per dose for a human subject, particularly an adult subject, wherein the second administration is about 6 months (about 180 days) after the first administration, and wherein the optional third administration (first booster dose) is about 18 months after the first administration.

Also alternatively, in one embodiment, the compositions described herein and methods of use thereof according to the present invention are characterized in that the total protein content of the three fusion proteins is 135 μg per dose for a human subject, particularly a pediatric subject, or may be only 67.5 μg per dose for a younger pediatric subject (in particular, a pediatric subject from birth to 4 years, such as 1-4 years old, or 2-4 years old), wherein the second administration is about 6 months (about 180 days) after the first administration, and wherein the optional third administration (first booster dose) is about 18 months after the first administration.

Alternatively, in a preferred embodiment, the compositions described herein and methods of use thereof according to the present invention are characterized in that the total protein content of the three fusion proteins is 180 μg per dose for a human subject, particularly an adult subject, wherein the second administration is about 6 months (about 180 days) after the first administration, and wherein the optional third administration (first booster dose) is about 18 months after the first administration.

Also alternatively, in a preferred embodiment, the compositions described herein and methods of use thereof according to the present invention are characterized in that the total protein content of the three fusion proteins is 180 μg per dose for a human subject, particularly a pediatric subject, or may be only 90 μg per dose for a younger pediatric subject (in particular, a pediatric subject from birth to 4 years, such as 1-4 years old, or 2-4 years old), wherein the second administration is about 6 months (about 180 days) after the first administration, and wherein the optional third administration (first booster dose) is about 18 months after the first administration.

Preferably, the methods and compositions for use according to the present invention are characterized in that the immune response elicited comprises an anti-OspA serotype 1, an anti-OspA serotype 2, an anti-OspA serotype 3, an anti-OspA serotype 4, anti-OspA serotype 5 and/or an anti-OspA serotype 6 antibody response. In some embodiments, antibodies of the immune response exhibit bactericidal activity (e.g., by promoting complement deposition on and/or phagocytosis of spirochetes bound by antibodies).

Preferably, the methods and compositions for use according to the present invention are characterized in that the immune response elicited comprises antibodies against spirochetes expressing OspA of serotypes 1, 2, 3, 4, 5, and/or 6. In some embodiments, the antibodies have bactericidal activity against spirochetes expressing OspA of serotypes 1, 2, 3, 4, 5, and/or 6.

Preferably, the methods and the compositions for use according to the present invention elicit an immune response comprising antibodies against Borrelia serotypes 1, 2, 3, 4, 5, and/or 6, wherein the immune response after administering at least three doses of the composition to an adult subject is higher than the immune response after administering at least two doses of the composition to an adult subject.

Preferably, the methods and the compositions for use according to the present invention elicit an immune response comprising antibodies against Borrelia serotypes 1, 2, 3, 4, 5, and/or 6, wherein the immune response after administering a second dose of the composition to a pediatric subject is at least as high as or higher than the immune response after administering a third dose of the composition to an adult subject.

Preferably, the methods and the compositions for use according to the present invention elicit an immune response comprising antibodies against Borrelia serotypes 1, 2, 3, 4, 5, and/or 6, wherein the immune response after administering at least three doses of the composition to a pediatric subject is higher than the immune response after administering at least three doses of the composition to an adult subject.

Preferably, the methods and compositions for use according to the present invention elicit an immune response comprising antibodies against Borrelia serotypes 1, 2, 3, 4, 5, and/or 6, wherein the immune response after administering at least two doses of the composition to a pediatric subject is higher than the immune response after administering at least two doses of the composition to an adult subject.

Preferably, the above compositions described herein and methods of use thereof according to the present invention are characterized in that the immune response against OspA serotypes 1, 2, 3, 4, 5, and 6 is sustained for at least about 60 days, for at least about 180 days, for at least about 365 days or for at least about 540 days.

In one embodiment, the composition or vaccine of the invention further comprises at least one additional antigen (herein referred to generically as “combination vaccine”). In a preferred embodiment, the at least one additional antigen is derived from a Borrelia species causing Lyme borreliosis. In various aspects, the at least one additional antigen is derived from another pathogen, preferably a tick-borne pathogen. In a further aspect, the pathogen causes Rocky Mountain spotted fever, Human granulocytic ehrlichiosis (HGE), Sennetsu Fever, Human Monocytic Ehrlichiosis (HME), Anaplasmosis, Boutonneuse fever, Rickettsia parkeri Rickettsiosis, Southern Tick-Associated Rash Illness (STARI), Helvetica Spotted fever, 364D Rickettsiosis, African spotted fever, Relapsing fever, Tularemia, Colorado tick fever, Tick-borne encephalitis (TBE, also known as FSME), Crimean-Congo hemorrhagic fever, Q fever, Omsk hemorrhagic fever, Kyasanur forest disease, Powassan encephalitis, Heartland virus disease or Babesiosis. In a further aspect, the disease is Japanese encephalitis. In a further embodiment, the at least one additional antigen is derived from a vector-borne, preferably a tick-borne, pathogen selected from the group comprising Borrelia hermsii, Borrelia parkeri, Borrelia duttoni, Borrelia miyamotoi, Borrelia turicatae, Rickettsia rickettsii, Rickettsia australis, Rickettsia conori, Rickettsia helvetica, Francisella tularensis, Anaplasma phagocytophilum, Ehrlichia sennetsu, Ehrlichia chaffeensis, Neoehrlichia mikurensis, Coxiella burnetii and Borrelia lonestari, Tick-borne encephalitis virus (TBEV aka FSME virus), Colorado tick fever virus (CTFV), Crimean-Congo hemorrhagic fever virus (CCHFV), Omsk Hemorrhagic Fever virus (OHFV), Japanese encephalitis virus (JEV) and Babesia spp.

Preferably, the compositions of the present invention are characterized in that the three fusion proteins comprise at least 60%, preferably at least 70%, more preferably at least 80% of all proteins in the composition. It is apparent to the person skilled in the art that the composition for use according to the present invention may also encompass further proteins different from the fusion proteins of SEQ ID Nos: 1, 2 and 3. However, the ratio of the fusion proteins should not be below the above limits, based on the weight. Other proteins may have a function, e.g. in stabilizing the composition or may be impurities.

In some embodiments, the compositions comprise three fusion proteins, preferably Lip-S1D1-S2D1 (SEQ ID NO: 1), Lip-S4D1-S3hybD1 (SEQ ID NO: 2) and Lip-S5D1-S6D1 (SEQ ID NO: 3) in a weight ratio of 1:1:1, 1:2:1, 1:3:1, 1:1:2, 1:1:3, 1:2:2, 1:2:3, 1:3:2, 1:3:3, 2:1:1, 2:1:2, 2:1:3, 2:2:3, 2:2:1, 2:3:1, 2:3:2, 2:3:3, 3:1:1, 3:1:2, 3:1:3, 3:2:1, 3:2:2, 3:2:3, 3:3:1, or 3:3:2. Preferably, the compositions according to the present invention are characterized in that the compositions comprise the fusion proteins in a weight ratio of 1:1:1 (Lip-S1 D1-S2D1:Lip-S4D1-S3hybD1:Lip-S5D1-S6D1).

Preferably, the compositions according to the present invention are characterized in that the compositions comprise an adjuvant, more preferably an aluminum adjuvant.

The choice of a suitable adjuvant to be mixed with bacterial proteins made using the processes of the invention is within the knowledge of the person skilled in the art. Suitable adjuvants include an aluminium salt such as aluminium hydroxide or aluminum phosphate, but may also be other metal salts such as those of calcium, magnesium, iron or zinc, or may be an insoluble suspension of acylated tyrosine, or acylated sugars, cationically or anionically derivatized saccharides, or polyphosphazenes. In a preferred embodiment, the composition is adjuvanted with an aluminium adjuvant, such as aluminium hydroxide. In a further preferred embodiment, the amount of copper associated in the aluminium adjuvant is less than 1.25 ppb in the vaccine composition.

In one embodiment, the compositions described herein and methods of use thereof according to the present invention comprise less than 1.25 ppb copper or a sufficient amount of L-methionine. Aluminum is an adjuvant often used in vaccination. The aluminum adjuvants typically comprise impurities, particularly heavy metals, such as copper, nickel and iron. The presence of these, particularly of coppers, lowers the bioavailability of the OspA protein in the vaccine. Without being bound to the theory it is assumed that the OspA protein binds to the aluminum and the heavy metal, particularly the copper, prevents the release, thus decreasing the bioavailability of the OspA protein in the vaccine. Therefore, the composition and in particular the aluminum adjuvant comprises less than 1.25 ppb copper. The unit ppb (part per billion) is often used in the field of mass spectrometry to quantify impurities. In case of aqueous solutions, 1 ppb means that 1 ng of substance (impurity) is present in 1 g solution, which means that 1 ppb equals 1 μg/l (assuming that 1 liter of solution has a weight of 1 kg). Typically, copper is in form of an ion, particularly as Cu+ or Cu²⁺. In a preferred embodiment, the composition comprises less than 1.00 ppb, less than 0.75 ppb or less than less than 0.50 ppb copper based on the weight of the aqueous composition. It is known that L-methionine is capable of binding to copper. The amount of L-methionine required will evidently depend from the amount of copper in the composition. The person skilled in the art will be capable of selecting a suitable amount of L-methionine. L-methionine may be used to complex excess copper in the composition for use in the present invention. A typical suitable concentration of L-methionine in the composition is at least 10 mmol/l. Depending on the amount of copper in the composition, the concentration may be even higher such as at least 20 mmol/l, at least 30 mmol/l, at least 40 mmol/l or at least 50 mmol/l or lower such as at most 10 mmol/l, at most 5 mmol/l or at most 1 mmol/l. Alternatively, the concentration of L-methionine is determined based on the concentration of copper in the composition. Particularly, the concentration of L-methionine in mol/d is at least equivalent with the concentration of copper in the composition. Alternatively, the concentration of L-methionine in mol/d is at least twice, threefold, fourfold, fivefold, or even tenfold, the concentration of copper in the composition.

In one embodiment, compositions described herein and methods of use thereof further comprise a reactive compound, wherein the reactive compound is selected from the group consisting of a redox active compound, a radical building compound, a stabilizing compound and a combination of any thereof. Without being bound to theory, antigen degradation of protein vaccines in aqueous compositions comprising heavy metal ions present in an aluminium salt, such as aluminium hydroxide, might be explained with an underlying degradation pathway assuming free-radicals such as e.g. free-radicals of sulphite. Heavy metal-catalysed oxidation is a degradation pathway resulting in covalent modification of proteins. The modified physicochemical properties of the oxidized/modified protein or antigen may result in loss of biological activity. Redox active compounds are suitable to prevent this modification. Preferably, the reactive compound is selected from the group consisting of formaldehyde, ethanol, chloroform, trichloroethylene, acetone, triton-X-100, deoxycholate, diethylpyrocarbonate, sulphite, Na2S2O5, beta-proprio-lacton, polysorbate such as Tween 20®, Tween 80®, 02, phenol, pluronic type copolymers, and a combination of any thereof.

Previous studies relating to vaccines, such as MBV (also referred to as Vaccine A), administered at a total protein content of the three fusion proteins at 90 μg, 135 μg and 180 μg per dose to adult subjects at a Month 0-1-2 and Month 0-2-6 is provided in PCT Publication WO 2021/207615, which is incorporated by reference herein in its entirety.

### ELISA

For the evaluation of immunogenicity described in the Examples, human sera is analyzed for IgG against each OspA serotype (ST1 to ST6) separately by a quantitative ELISA. Dilution series of sera are added to microtiter plates coated with full length OspA ST1, ST2, ST3, ST4, ST5 or ST6. Presence of binding IgGs are detected with an anti-human IgG enzyme conjugate followed by addition of the substrate. The optical density of the colored end product is proportional to the amount of protein specific IgG present in the serum that can be quantified on the basis of the Reference Substance curve by four-parameter logistic fit and parallel line analysis.

### FURTHER EMBODIMENTS OF THE PRESENT INVENTION

1. A composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins in the range of from 60 μg to 200 μg per dose for use in a method of eliciting an immune response protective against Lyme disease in a subject, wherein the subject is administered at least three doses of the composition.
2. A composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose for use in a method for treating, preventing or reducing the risk of Lyme disease, wherein the subject is administered at least three doses of the composition.
3. A composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose for use in a method of vaccinating a human subject against Lyme disease, wherein the subject is administered at least three doses of the composition.
4. The composition of any one of embodiments 1 to 3, wherein a second dose is administered in a period of at least 6 weeks to at most 3 months after a first dose is administered, and a third dose is administered in a period of at least 5 months to at most 9 months or at least 5 months to at most 7 months after the first dose is administered.
5. The composition of any one of embodiments 1 to 4, wherein a second dose is administered in a period of at least 50 days to at most 70 days after a first dose is administered, and a third dose is administered in a period of at least 170 days to at most 190 days after the first dose is administered.
6. The composition of any one of embodiments 1 to 5, wherein a first dose is administered on day 1, a second dose is administered about 2 months after the first dose, and a third dose is administered about 6 months after the first dose is administered.
7. The composition of any one of embodiments 1 to 6, wherein a fourth dose is administered in the period of at least 15 months to at most 21 months, particularly in the period of at least 17 months to at most 19 months after the first dose is administered or about 12 months after the third administration.
8. The composition of any one of embodiments 1 to 7, wherein a fourth dose is administered about 18 months after the first dose is administered or about 12 months after the third administration.
9. The composition of any one of embodiments 1 to 8, wherein the subject is aged 5 years or older, such as 5-65 years.
10. The composition of any one of embodiments 1 to 9, wherein the subject is an adult subject aged 18 years or older, such as 18-65 years and/or 50 years or older.
11. The composition of any one of embodiments 1 to 9, wherein the subject a pediatric subject from birth to 17 years.
12. The composition of embodiment 11, wherein the pediatric subject is aged 5-17 years, such as 5-11 years and/or 12-17 years.
13. The composition of embodiment 11, wherein the pediatric subject is from birth to 4 years, such as 1-4 years and/or 2-4 years.
14. The composition of any one of embodiments 1 to 13, wherein the total protein content of the three fusion proteins is 135 μg or 180 μg per dose.
15. The composition of any one of embodiments 1 to 13, wherein the total protein content of the three fusion proteins is 67.5 μg or 90 μg per dose.
16. The composition of any one of embodiments 1 to 15, wherein the three fusion proteins comprise at least 60%, preferably at least 70%, more preferably at least 80% of all proteins in the composition.
17. The composition of any one of embodiments 1 to 16, wherein the composition comprises the fusion proteins in a weight ratio of 1:1:1 (Lip-S1D1-S2D1:Lip-S4D1-S3hybD1:Lip-S5D1-S6D1).
18. The composition of any one of embodiments 1 to 17, wherein the total protein content of the three fusion proteins is 180 μg per dose, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.
19. The composition of any one of embodiments 1 to 18, wherein the subject is an adult subject, wherein the total protein content of the three fusion proteins is 180 μg per dose, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.
20. The composition of any one of embodiments 1 to 19, wherein the subject is a pediatric subject, wherein the total protein content of the three fusion proteins is 180 μg per dose, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.
21. The composition of any one of embodiments 1 to 20, wherein the subject is a pediatric subject from birth to 4 years, wherein the total protein content of the three fusion proteins is 67.5 μg or 90 μg per dose, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.
22. The composition of any one of embodiments 1 to 21, wherein a further dose is administered every year after the third dose or the optional fourth dose is administered, particularly after 1 year, after 2 years, and after 3 years.
23. A composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose for use in a method of eliciting an immune response protective against Lyme disease in a subject, wherein the subject is administered at least two doses of the composition.
24. A composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose for use in a method for treating, preventing or reducing the risk of Lyme disease, wherein the subject is administered at least two doses of the composition.
25. A composition comprising a fusion protein of SEQ ID NO: 1 (Lip-S1 D1-S2D1), a fusion protein of SEQ ID NO: 2 (Lip-S4D1-S3hybD1) and a fusion protein of SEQ ID NO: 3 (Lip-S5D1-S6D1) at a total protein content of the three fusion proteins in the range of from 60 μg to 200 μg per dose for use in a method of vaccinating a human subject against Lyme disease, wherein the subject is administered at least two doses of the composition.
26. The composition of any one of embodiments 23 to 25, wherein a second dose is administered in a period of at least 5 months to at most 7 months after a first dose is administered.
27. The composition of any one of embodiments 23 to 26, wherein a second dose is administered in a period of at least 170 days to at most 190 days after a first dose is administered.
28. The composition of any one of embodiments 23 to 27, wherein a first dose is administered on day 1 and a second dose is administered about 6 months after the first dose is administered.
29. The composition of any one of embodiments 23 to 28, wherein a third dose is administered in the period of at least 15 months to at most 21 months, particularly in the period of at least 17 months to at most 19 months after the first dose is administered.
30. The composition of any one of embodiments 23 to 29, wherein a third dose is administered about 18 months after the first dose is administered.
31. The composition of any one of embodiments 23 to 30, wherein the subject is aged 5 year or older, such as 5-65 years.
32. The composition of any one of embodiments 23 to 31, wherein the subject is an adult subject aged 18 years or older, such as 18-65 years and/or 50 years or older.
33. The composition of any one of embodiments 23 to 31, wherein the subject is a pediatric subject from birth to 17 years.
34. The composition of embodiment 33, wherein the pediatric subject is aged 5-17 years, such as 5-11 years and/or 12-17 years.
35. The composition of embodiment 33, wherein the pediatric subject is from birth to 4 years, such as 1-4 years and/or 2-4 years.
36. The composition of any one of embodiments 23 to 35, wherein the total protein content of the three fusion proteins is 135 μg or 180 μg per dose.
37. The composition of any one of embodiments 23 to 35, wherein the total protein content of the three fusion proteins is 67.5 μg or 90 μg per dose.
38. The composition of any one of embodiments 23 to 37, wherein the three fusion proteins comprise at least 60%, preferably at least 70%, more preferably at least 80% of all proteins in the composition.
39. The composition of any one of embodiments 23 to 38, wherein the composition comprises the three fusion proteins in a weight ratio of 1:1:1 (Lip-S1 D1-S2D1:Lip-S4D1-S3hybD1:Lip-S5D1-S6D1).
40. The composition of any one of embodiments 23 to 39, wherein the total protein content of the three fusion proteins is 180 μg per dose, wherein a first dose is administered on day 1, a second dose is administered in a period of at least 170 days to at most 190 days after the first dose, and wherein optionally a third dose is administered about 18 months after the first dose is administered.
41. The composition of any one of embodiments 23 to 40, wherein the subject is an adult subject, wherein the total protein content of the three fusion proteins is 180 μg per dose, wherein a first dose is administered on day 1, a second dose is administered in a period of at least 170 days to at most 190 days after the first dose, and wherein optionally a third dose is administered about 18 months after the first dose is administered.
42. The composition of any one of embodiments 23 to 41, wherein the subject is a pediatric subject, wherein the total protein content of the three fusion proteins is 180 μg per dose, wherein a first dose is administered on day 1, a second dose is administered in a period of at least 170 days to at most 190 days after the first dose, and wherein optionally a third dose is administered about 18 months after the first dose is administered.
43. The composition of any one of embodiments 23 to 42, wherein the subject is a pediatric subject from birth to 4 years, wherein the total protein content of the three fusion proteins is 67.5 μg or 90 μg per dose, wherein a first dose is administered on day 1, a second dose is administered in a period of at least 170 days to at most 190 days after the first dose, and wherein optionally a third dose is administered about 18 months after the first dose is administered.
44. The composition of any one of embodiments 23 to 43, wherein a further dose is administered every year after the second dose or the optional third dose is administered, particularly after 1 year, after 2 years, and after 3 years.
45. The composition of any one of embodiments 1 to 44, wherein the composition elicits an immune response comprising an anti-OspA serotype 1, an anti-OspA serotype 2, an anti-OspA serotype 3, an anti-OspA serotype 4, anti-OspA serotype 5 and/or an anti-OspA serotype 6 antibody response.
46. The composition of any one of embodiments 1 to 45, wherein the composition elicits an immune response comprising antibodies against Borrelia serotypes 1, 2, 3, 4, 5, and 6.
47. The composition of any one of embodiments 1 to 46, wherein the composition elicits an immune response comprising antibodies against Borrelia serotypes 1, 2, 3, 4, 5, and/or 6, wherein the immune response after administering at least three doses of the composition to an adult subject is higher than the immune response after administering at least two doses of the composition to an adult subject.
48. The composition of embodiment 47, wherein geometric mean titers (GMTs) of antibodies against Borrelia serotypes after administering at least three doses of the composition to an adult subject are at least 2.0-fold higher than the GMTs after administering at least two doses of the composition to an adult subject.
49. The composition of embodiment 48, wherein the GMTs of antibodies against Borrelia serotypes after administering at least three doses of the composition to an adult subject are about 2.0-fold to about 3.0-fold-higher than the GMTs after administering at least two doses of the composition to an adult subject.
50. The composition of any one of embodiments 1 to 49, wherein the composition elicits an immune response comprising antibodies against Borrelia serotypes 1, 2, 3, 4, 5, and/or 6, wherein the immune response after administering a second dose of the composition to a pediatric subject is at least as high as or higher than the immune response after administering a third dose of the composition to an adult subject.
51. The composition of any one of embodiments 1 to 50, wherein the composition elicits an immune response comprising antibodies against Borrelia serotypes 1, 2, 3, 4, 5, and/or 6, wherein the immune response after administering at least three doses of the composition to a pediatric subject is higher than the immune response after administering at least three doses of the composition to an adult subject.
52. The composition of embodiment 51, wherein geometric mean titers (GMTs) of antibodies against Borrelia serotypes after administering at least three doses of the composition to a pediatric subject are at least 2.0-fold higher than the GMTs after administering at least three doses of the composition to an adult subject.
53. The composition of embodiment 52, wherein the GMTs of antibodies against Borrelia serotypes after administering at least three doses of the composition to a pediatric subject aged 12-17 years are about 2.0-fold to about 3.0-fold higher that the GMTs after administering at least three doses of the composition to an adult subject.
54. The composition of embodiment 52, wherein the GMTs of antibodies against Borrelia serotypes after administering at least three doses of the composition to a pediatric subject aged 5-11 years are about 3.0-fold to about 5.0-fold higher that the GMTs after administering at least three doses of the composition to an adult subject.
55. The composition of any one of embodiments 1 to 54, wherein the composition elicits an immune response comprising antibodies against Borrelia serotypes 1, 2, 3, 4, 5, and/or 6, wherein the immune response after administering at least two doses of the composition to a pediatric subject is higher than the immune response after administering at least two doses of the composition to an adult subject.
56. The composition of embodiment 55, wherein geometric mean titers (GMTs) of antibodies against Borrelia serotypes after administering at least two doses of the composition to a pediatric subject are at least 2.0-fold higher than the GMTs after administering at least two doses of the composition to an adult subject.
57. The composition of embodiment 56, wherein the GMTs of antibodies against Borrelia serotypes after administering at least two doses of the composition to a pediatric subject aged 12-17 years are about 4.0-fold to about 6.0-fold higher that the GMTs after administering at least two doses of the composition to an adult subject.
58. The composition of embodiment 56, wherein the GMTs of antibodies against Borrelia serotypes after administering at least two doses of the composition to a pediatric subject aged 5-11 years are about 5.0-fold to about 8.0-fold higher than the GMTs after administering at least two doses of the composition to an adult subject.
59. The composition of any one of embodiments 1 to 58, wherein the composition elicits an immune response comprising antibodies against Borrelia serotypes 1, 2, 3, 4, 5, and 6 that is sustained for at least about 60 days, for at least about 180 days, for at least about 365 days, or for at least about 540 days.
60. The composition of embodiment 59, wherein the immune response is sustained above baseline for at least about 180 days after administering at least two doses or at least three doses of the composition.
61. The composition of embodiment 60, wherein geometric mean fold rises (GMFRs) of antibodies against Borrelia serotypes at least 180 days after administering at least three doses of the composition to a pediatric subject aged 5-11 are at least 2.0-fold higher than the GMFRs at baseline.
62. The composition of embodiment 61, wherein the GMFRs of antibodies against Borrelia serotypes at least 180 days after administering at least three doses of the composition to a pediatric subject aged 5-11 are about 2.0-fold to 7.0-fold higher than the GMFRs at baseline.
63. The composition of any one of embodiments 1 to 62, wherein the composition is administered to a subject in a volume of 0.25 ml, 0.5 ml, or 1.0 ml.
64. The composition of any one of embodiments 1 to 63, wherein the composition is administered to a subject in a volume of 0.5 ml.
65. The composition of any one of embodiments 1 to 64, wherein the composition comprises at least one of sodium phosphate, sodium chloride, sucrose and polysorbate 20.
66. The composition of any one of embodiments 1 to 65, wherein the sodium phosphate is present at a concentration between 5 mM and 50 mM, the sodium chloride is present at a concentration between 100 mM and 200 mM, the sucrose is present at a concentration between 2.5% and 10% and the polysorbate 20 is present at a concentration between 0.01% and 0.1%.
67. The composition of any one of embodiments 1 to 66, wherein the composition comprises an adjuvant.
68. The composition of embodiment 67, wherein the adjuvant comprises an aluminum adjuvant.
69. The composition of any one of embodiments 1 to 68, wherein the composition comprises less than 1.25 ppb copper.
70. The composition of embodiment 69, wherein the copper is in the form of an ion, particularly as Cu+ or Cu2+.
71. The composition of any one of embodiments 1 to 70, wherein the composition comprises L-methionine.
72. The composition of embodiment 71, wherein the L-methionine is present in a concentration of at least 10 mmol/l.
73. The composition of any one of embodiments 69 to 72, wherein the concentration of L-methionine in mol/I is at least equivalent with the concentration of copper in the composition.
74. The composition of any one of embodiments 1 to 73, wherein the composition further comprises a reactive compound, wherein the reactive compound is selected from the group consisting of a redox active compound, a radical building compound, a stabilizing compound and a combination of any thereof, especially wherein the reactive compound is selected from the group consisting of formaldehyde, ethanol, chloroform, trichloroethylene, acetone, Triton-X-100, deoxycholate, diethylpyrocarbonate, sulphite, Na2S2O5, beta-proprio-lacton, polysorbate such as Tween 20®, Tween 80®, 02, phenol, pluronic type copolymers, and a combination of any thereof.
75. The composition of any one of embodiments 1 to 74, for use in a method of eliciting an immune response protective against Lyme disease in a subject.
76. The composition of any one of embodiments 1 to 75, for use in a method of treating, preventing or reducing the risk of Lyme disease in a subject.
77. The composition of any one of embodiments 1 to 76, for use in a method of vaccinating a subject against Lyme disease.

### EXAMPLES

### Example 1. Phase 2 Clinical Trial with Multivalent Borrelia Vaccine (MBV) Investigating Three-Dose or Two-Dose Primary Immunization Schedule of the Vaccine, and Booster Dose, in a Study Population Aged 5 to 65 Years

This study is a randomized, observer-blind, placebo controlled, multicenter Phase 2 study in healthy subjects aged 5 to 65 years. This study is conducted to compare a three and two dose primary immunization schedule (Month 0-2-6 or Month 0-6) in adults (aged 18-65 years), adolescents (aged 12-17 years) and children (aged 5-11 years). In this study, MBV is administered at a dose of 180 μg total protein (i.e., 60 μg per fusion protein) and aluminum hydroxide adjuvant (0.5 mg) in 0.5 mL of buffer. All injections are administered intramuscularly (I.M.). This study evaluates the immune responses to a booster dose administered about 12 months after completion of the extended primary vaccination schedule and immunopersistence for up to 3 years following the booster dose.

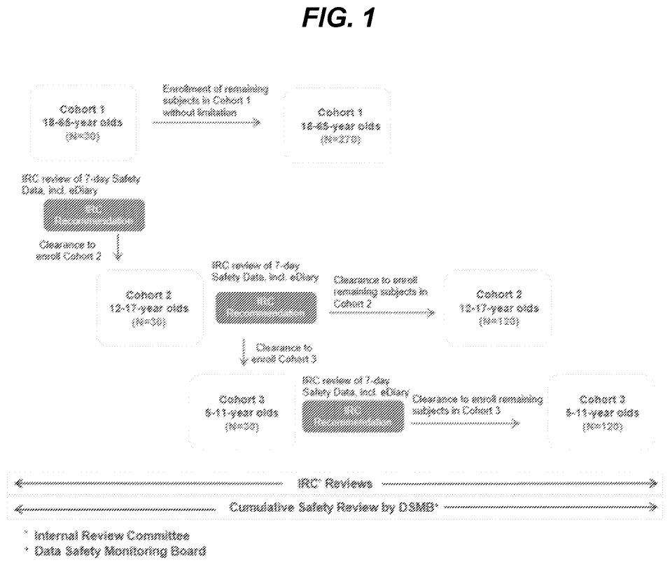

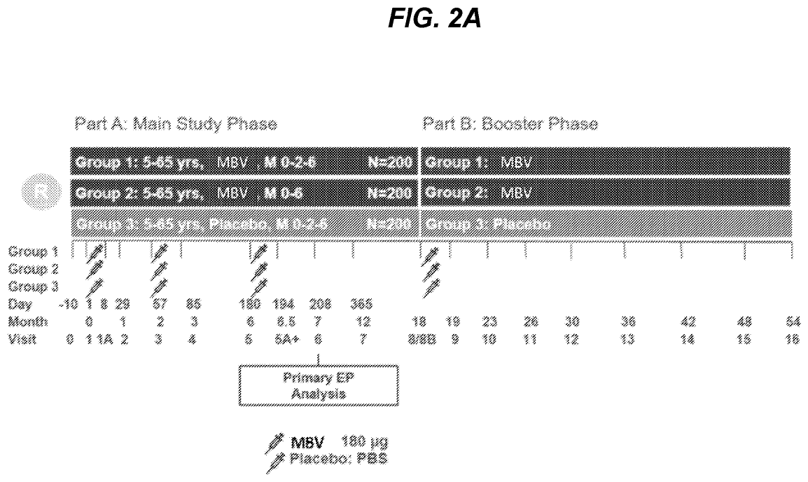

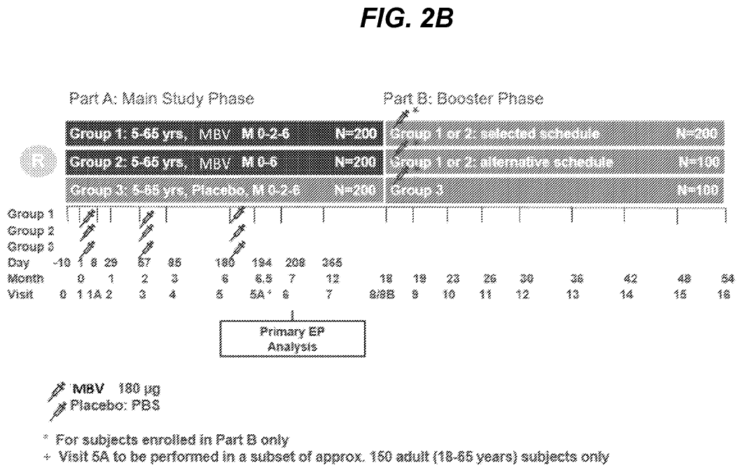

Enrollment is performed in an age-descending, staggered manner for the three age cohorts (see FIG. 1) and the study is conducted in two study parts-Part A: Main Study Phase, Part B: Booster Phase (see FIG. 2A and FIG. 2B).

As shown in Table 1, in Part A (Main Study Phase) a total of approximately 600 subjects aged 5 to 65 years are randomized 1:1:1 into three study groups: Group 1 (approximately 200 subjects) receives three vaccinations of MBV at Month 0-2-6. Group 2 (approximately 200 subjects) receives two MBV vaccinations at Month 0-6 and a placebo injection at Month 2 in order to keep the blind. Group 3 (approximately 200 subjects) receives three placebo injections at Month 0-2-6. Within each study group subjects are enrolled 2:1:1 in three age cohorts (18-65 years, 12-17 years and 5-11 years).

In Part A, all subjects receive three I.M. vaccinations at Month 0-2-6 (i.e., Day 1-57-180). On Day 8/Visit 1A (i.e., 7 days after the first vaccination) a safety visit is performed (phone call for subjects aged 18-65, in-person visit for subjects aged 5-17 years). In-person visits are scheduled for all age cohorts one month after each vaccination. Blood samples for immunogenicity assessments are collected at the screening visit, Day 85, Day 180, Day 194 (in a subset of adult subjects), Day 208, Day 365/Month 12 and at Month 18.

In Part B (Booster Phase), all eligible subjects will receive a booster dose of MBV or placebo injection at Month 18.

TABLE 1
Study Groups and Vaccinations for Part A (Main Study Phase)

| | | | Age Cohort | | |
|---|---|---|---|---|---|
| | Study | | (Age in years | | Vaccination |
| | Group | Subjects | at screening) | Treatment | Schedule |
| Part A: | Group 1 | Total: | 18-65 | MBV 180 μg | Month 0-2-6 |
| Main | | 200 | 12-17 | | |
| Study | | 100 | 5-11 | | |
| Phase | | 50 | | | |
| | | 50 | | | |
| | Group 2 | Total: | 18-65 | MBV 180 μg | Month 0-6 |
| | | 200 | 12-17 | Placebo | Month 2* |
| | | 100 | 5-11 | | |
| | | 50 | | | |
| | | 50 | | | |
| | Group 3 | Total: | 18-65 | Placebo | Month 0-2-6 |
| | | 200 | 12-17 | | |
| | | 100 | 5-11 | | |
| | | 50 | | | |
| | | 50 | | | |
| Part B: | Group 1 | Total: | 18-65 | MBV 180 μg | Month 18 |
| Booster | | 200 | 12-17 | | |
| Phase | | 100 | 5-11 | | |
| | | 50 | | | |
| | | 50 | | | |
| | Group 2 | Total: | 18-65 | MBV 180 μg | Month 18 |
| | | 200 | 12-17 | | |
| | | 100 | 5-11 | | |
| | | 50 | | | |
| | | 50 | | | |
| | Group 3 | Total: | 18-65 | Placebo | Month 18 |
| | | 200 | 12-17 | | |
| | | 100 | 5-11 | | |
| | | 50 | | | |
| | | 50 | | | |

*In order to keep the blind, subjects assigned to Group 2 will receive a sham injection of placebo at Month 2.

### Immunogenicity Analysis

The primary immunogenicity objective of this study is to assess the immune response to MBV, applied in a three or two dose primary immunization schedule (Month 0-2-6 or Month 0-6), in a healthy study population aged 5 to 65 years at Day 208 (Month 7). The primary immunogenicity endpoint is Geometric Mean Titers (GMTs) for IgG against each OspA serotype ST1 to ST6, determined by an ELISA assay, at Day 208/Month 7 (Part A).

The primary immunogenicity analysis is an overall and group-wise comparison of the OspA serotype specific IgG GMTs in the per-protocol analysis set between study groups at Day 208 (i.e. 28 days after the last vaccination) by ANOVA (factors treatment group, age group). In addition, GMTs and GMFRs (i.e., increase of IgG titers compared to respective titers at Day 1 (baseline)) against each OspA serotype ST1 to ST6 are compared overall and pair-wise between study groups on specified time points.

Immunogenicity assessments measuring OspA serotype specific IgG by an IgG binding assay are performed on samples collected at Visit 0 and Visit 4 to 8 of the Main Study Phase (Part A) and at Visit 9 to 16 during the Booster Phase (Part B).

### Immunogenicity Analysis: Day 208/Month 7

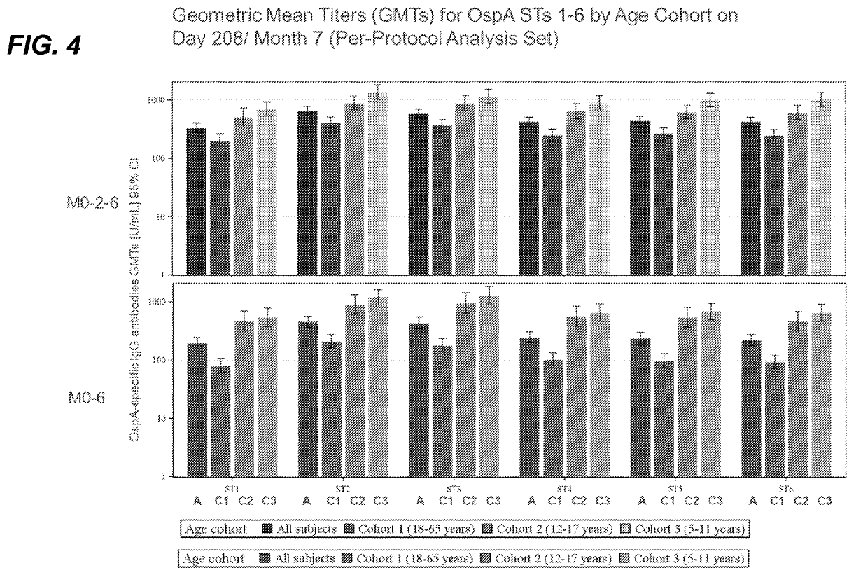

Human sera was analyzed for IgG against each OspA serotype (ST1 to ST6) separately by a quantitative ELISA. FIG. 3 shows the GMTs for OspA serotypes (ST1 to ST6) on Day 208/Month 7 for adults (18-65 years), adolescents (12-17 years) and children (5-11 years) receiving either the three dose MBV (M 0-2-6), two dose MBV (M 0-6) or placebo. FIG. 4 shows GMTs for OspA serotypes (ST1 to ST6) by age cohort on Day 208/Month 7 receiving either the three dose MBV (M 0-2-6) or two dose MBV (M 0-6). See Table 3 below.

FIG. 5 shows the GMTs for OspA serotypes (ST1 to ST6) over time (Visit 1-Month 0/Screening, Visit 4-Month 4/Day 85, Visit 5-Month 6/Day 180, Visit 5A-Month 6/Day 194 (adults 18-65 years only) and Visit 6-Month 7/Day 208) for each age cohort of Group 1 (3 three dose MBV, M 0-2-6). In adolescents (12-17 years), GMTs for all six serotypes after two MBV doses at M 0-2 are as high as for adults (18-65 years) after three doses at M 0-2-6. In children (5-11 years), GMTs for all six serotypes after two MBV doses at M 0-2 are higher than for adults (18-65 years) after three doses at M 0-2-6.

Table 2 shows the GMTs for OspA-specific IgG by serotype on Day 208/Month 7. Overall, GMTs ranged from 332.9 [ST1] to 657.7 [ST2] in the M 0-2-6 schedule group and from 195.7 [ST1] to 456.6 [ST2] in the M 0-6 schedule group. In adults (18-65 years), GMTs ranged from 199.1 [ST1] to 418.5 [ST2] in the M 0-2-6 schedule group and from 80.9 [ST1] to 211.5 [ST2] in the M 0-6 schedule group. In adolescents (12-17 years), GMTs ranged from 513.8 [ST1] to 900.0 [ST2] in the M 0-2-6 schedule group and from 463.3 [ST6] to 961.3 [ST3] in the M 0-6 schedule group. In children (5-11 years), GMTs ranged from 706.6 [ST1] to 1364.1 [ST2] in the M 0-2-6 schedule group and from 543.3 [ST1] to 1293.1 [ST3] in the M 0-6 schedule group.

TABLE 2
GMTs for OspA-specific IgG by Serotype on Day 208/Month 7

| | | 180 ug MBV | | | |
|---|---|---|---|---|---|
| | | Group 1 | Group 2 | | |
| | | M 0-2-6 | M 0-6 | Placebo | |
| | | [U/mL] [95% CI] | [U/mL] [95% CI] | [U/mL] [95% CI] | |
| | | N(5-65 yrs) = 177 | N(5-65 yrs) = 162 | N(5-65 yrs) = 177 | |
| | | N(18-65 yrs) = 96 | N(18-65 yrs) = 84 | N(18-65 yrs) = 94 | |
| | | N(12-17 yrs) = 41 | N(12-17 yrs) = 40 | N(12-17 yrs) = 42 | p-value |
| Serotype | Age | N(5-11 yrs) = 40 | N(5-11 yrs) = 38 | N(5-11 yrs) = 41 | overall |
| ST1 | 5-65 yrs | 332.9 | 195.7 | 23.8 | <.0001 |
| | | [273.8, 404.7] | [154.8, 247.4] | [21.6, 26.2] | |
| | 18-65 yrs | 199.1 | 80.9 | 24.9 | <.0001 |
| | | [151.4, 261.8] | [62.2, 105.1] | [21.7, 28.6] | |
| | 12-17 yrs | 513.8 | 467.5 | 22.0 | <.0001 |
| | | [364.9, 723.4] | [313.8, 696.5] | [19.7, 24.6] | |
| | 5-11 yrs | 706.6 | 543.3 | 23.3 | <.0001 |
| | | [537.6, 928.6] | [377.0, 782.9] | [18.3, 29.8] | |
| ST2 | 5-65 yrs | 657.7 | 456.6 | 21.9 | <.0001 |
| | | [560.2, 772.2] | [367.3, 567.6] | [20.2, 23.7] | |
| | 18-65 yrs | 418.5 | 211.5 | 22.2 | <.0001 |
| | | [339.3, 516.2] | [162.8, 274.8] | [19.9, 24.8] | |
| | 12-17 yrs | 900.0 | 906.9 | 20.7 | <.0001 |
| | | [693.0, 1168.9] | [619.1, 1328.6] | [19.3, 22.2] | |
| | 5-11 yrs | 1364.1 | 1202.6 | 22.4 | <.0001 |
| | | [1030.9, 1804.9] | [883.5, 1637.2] | [17.8, 28.1] | |
| ST3 | 5-65 yrs | 589.0 | 432.5 | 21.9 | <.0001 |
| | | [497.9, 696.7] | [341.6, 547.6] | [20.2, 23.9] | |
| | 18-65 yrs | 369.5 | 179.2 | 21.9 | <.0001 |
| | | [296.8, 460.0] | [135.8, 236.4] | [19.5, 24.6] | |
| | 12-17 yrs | 884.4 | 961.3 | 21.3 | <.0001 |
| | | [654.1, 1195.8] | [650.2, 1421.3] | [19.8, 22.9] | |
| | 5-11 yrs | 1150.2 | 1293.1 | 22.6 | <.0001 |
| | | [865.6, 1528.5] | [927.4, 1802.9] | [17.7, 28.8] | |
| ST4 | 5-65 yrs | 423.3 | 243.6 | 23.5 | <.0001 |
| | | [354.4, 505.4] | [194.2, 305.5] | [21.3, 25.9] | |
| | 18-65 yrs | 252.7 | 103.3 | 23.9 | <.0001 |
| | | [199.3, 320.5] | [80.2, 133.2] | [20.8, 27.4] | |
| | 12-17 yrs | 644.5 | 568.2 | 22.8 | <.0001 |
| | | [479.3, 866.6] | [385.2, 838.1] | [20.3, 25.7] | |
| | 5-11 yrs | 913.9 | 653.9 | 23.2 | <.0001 |
| | | [697.4, 1197.7] | [463.0, 923.7] | [18.1, 29.9] | |
| ST5 | 5-65 yrs | 442.9 | 237.1 | 22.4 | <.0001 |
| | | [373.9, 524.7] | [188.5, 298.3] | [20.6, 24.4] | |
| | 18-65 yrs | 266.8 | 98.5 | 22.4 | <.0001 |
| | | [214.3, 332.2] | [75.8, 128.0] | [20.1, 25.0] | |
| | 12-17 yrs | 627.8 | 543.5 | 21.3 | <.0001 |
| | | [477.7, 825.1] | [372.0, 794.1] | [19.5, 23.3] | |
| | 5-11 yrs | 1006.1 | 681.1 | 23.7 | <.0001 |
| | | [761.8, 1328.8] | [488.5, 949.6] | [18.4, 30.4] | |
| ST6 | 5-65 yrs | 426.5 | 219.9 | 24.3 | <.0001 |
| | | [356.3, 510.5] | [175.6, 275.3] | [22.2, 26.7] | |
| | 18-65 yrs | 249.2 | 93.9 | 23.5 | <.0001 |
| | | [196.5, 316.0] | [73.1, 120.7] | [20.9, 26.3] | |
| | 12-17 yrs | 613.2 | 463.3 | 24.6 | <.0001 |
| | | [465.9, 807.1] | [314.7, 682.0] | [21.0, 28.8] | |
| | 5-11 yrs | 1025.0 | 650.9 | 26.1 | <.0001 |
| | | [769.0, 1366.1] | [466.4, 908.4] | [19.8, 34.3] | |

In adults, GMTs for all six serotypes were statistically significantly higher in the M 0-2-6 vs. the M 0-6 group at Day 208/Month 7, see Table 3. Immune responses in adolescents and children were comparable for both primary vaccination schedules. In general, GMTs for all six serotypes were higher in adolescents and children in comparison to adults. In adolescents GMTs were 2.2-fold [ST2] to 2.6-fold [ST1/ST4](three doses) and 4.3-fold [ST2] to 5.8-fold [ST1](two doses) higher than in adults. In children, GMTs were 3.1-fold [ST3] to 4.1-fold [ST6](three doses) and 5.7-fold [ST2] to 7.2-fold [ST3](two doses) higher than in adults. P-value are from pairwise comparisons which come from ANOVA analyses (ELISA: ANOVA (Factors: Study Group, Age Cohort) for GMT of OspAST1-6 Specific IgG Antibodies by Visit.

TABLE 3
Statistical significance in GMTs

| Statistical | | |
|---|---|---|
| significant | | |
| comparison | GM ratio | |
| between MBV | estimate | |
| groups | [95% CI] | p-value |
| Study Group 1 vs. | 18-65 yrs: | ST1, |
| Study Group 2 | ST1: 2.5 [1.7, 3.6]; | ST2: <.0001 |
| | ST2: 2.0 [1.4, 2.8] | |
| Study Group 1 vs. | 18-65 yrs: | ST3, |
| Study Group 2 | ST3: 2.1 [1.4, 2.9]; | ST4: <.0001 |
| | ST4: 2.4 [1.7, 3.5] | |
| Study Group 1 vs. | 18-65 yrs: | ST5, |
| Study Group 2 | ST5: 2.7 [1.9, 3.8]; | ST6: <.0001 |
| | ST6: 2.7 [1.9, 3.8] | |

Table 4 shows a comparison of GMTs for three and two dose vaccination schedules (M 0-2-6 and M 0-6) and different age groups. In younger age groups (adolescents 12-17 years and children 5-11 years), already after second dose of a M 0-2-6 schedule (i.e. at Day 85), the immune responses for all six serotypes were at least as high as after three doses (i.e. at Day 208) in adults 18-65 years.

TABLE 4
Comparison of GMTs for different vaccination schedules and age groups

| | M 0-2-6 schedule | | | M 0-6 schedule | |
|---|---|---|---|---|---|
| | M 0-2-6 | M 0-2 | M 0-2 | M 0-6 | M 0-6 |
| | Day 208/Month 7 | Day 85/Month 3 | Day 85/Month 3 | Day 208/Month 7 | Day 208/Month 7 |
| | Adults | Adolescents | Children | Adolescents | Children |
| | N(18-65 yrs) = 96 | N(12-17 yrs) = 41 | N(5-11 yrs) = 40 | N(12-17 yrs) = 40 | N(5-11 yrs) = 38 |
| Serotype | [U/mL] [95% CI] | [U/mL] [95% CI] | [U/mL] [95% CI] | [U/mL] [95% CI] | [U/mL] [95% CI] |
| ST1 | 199.1 | 198.5 | 269.5 | 467.5 | 543.3 |
| | [151.4, 261.81 | [138.4, 284.9] | [219.8, 330.5] | [313.8, 696.5] | [377.0, 782.9] |
| ST2 | 418.5 | 413.7 | 625.2 | 906.9 | 1202.6 |
| | [339.3, 516.2] | [286.7, 596.9] | [482.4, 810.3] | [619.1, 1328.6] | [883.5, 1637.2] |
| ST3 | 369.5 | 432.4 | 649.6 | 961.3 | 1293.1 |
| | [296.8, 460.0] | [294.9, 633.9] | [494.7, 853.1] | [650.2, 1421.3] | [927.4, 1802.9] |
| ST4 | 252.7 | 256.2 | 354.9 | 568.2 | 653.9 |
| | [199.3, 320.5] | [182.5, 359.6] | [275.2, 457.7] | [385.2, 838.1] | [463.0, 923.7] |
| ST5 | 266.8 | 270.6 | 441.2 | 543.5 | 681.1 |
| | [214.3, 332.2] | [190.0, 385.3] | [339.3, 573.7] | [372.0, 794.1] | [488.5, 949.6] |
| ST6 | 249.2 | 227.3 | 380.4 | 463.3 | 650.9 |
| | [196.5, 316.0] | [157.9, 327.1] | [293.3, 493.5] | [314.7, 682.0] | [466.4, 908.4] |

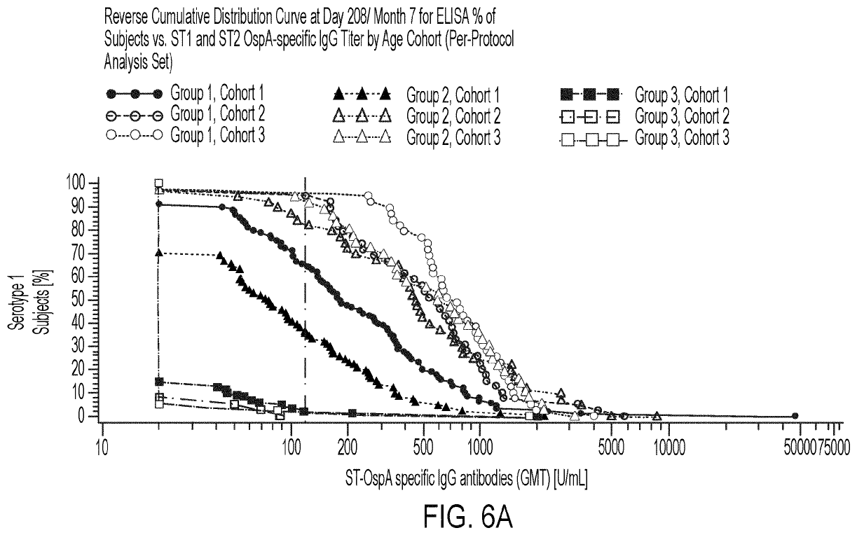

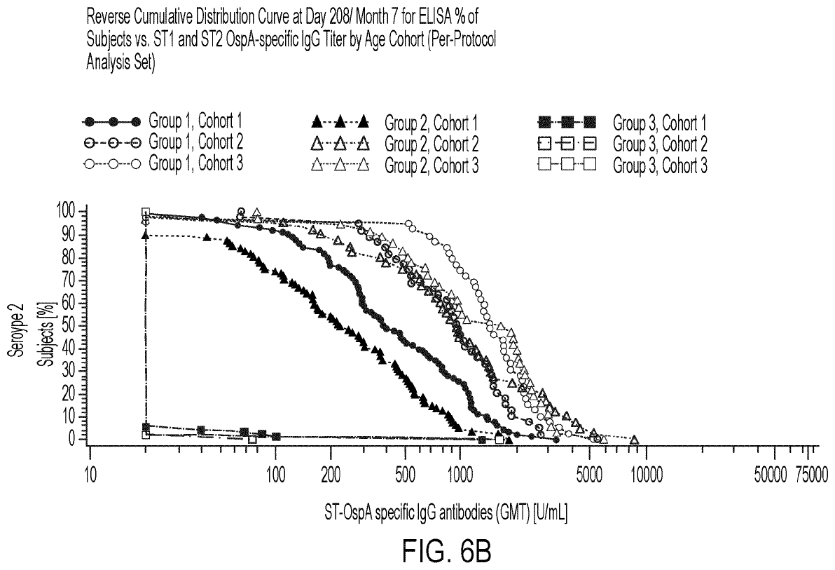

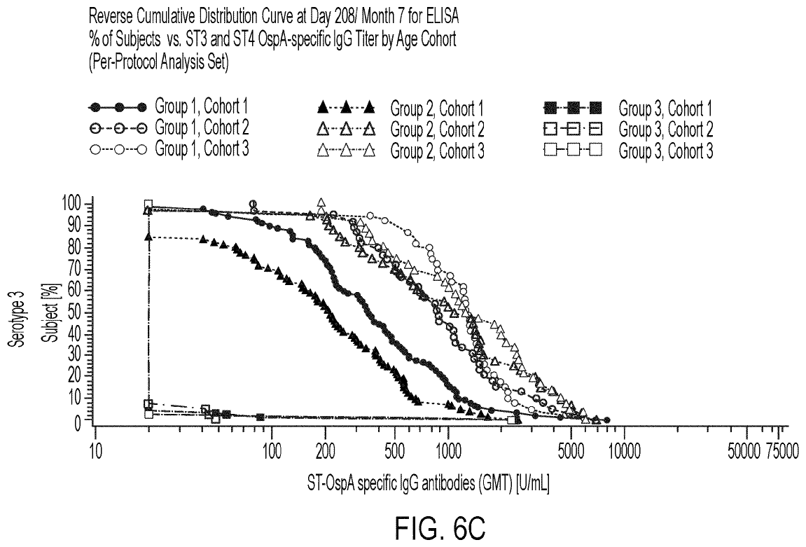

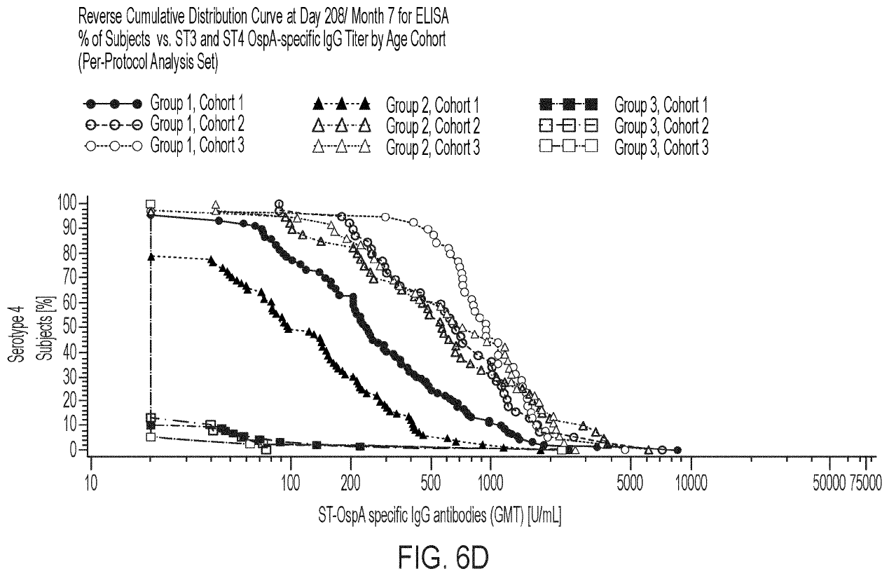

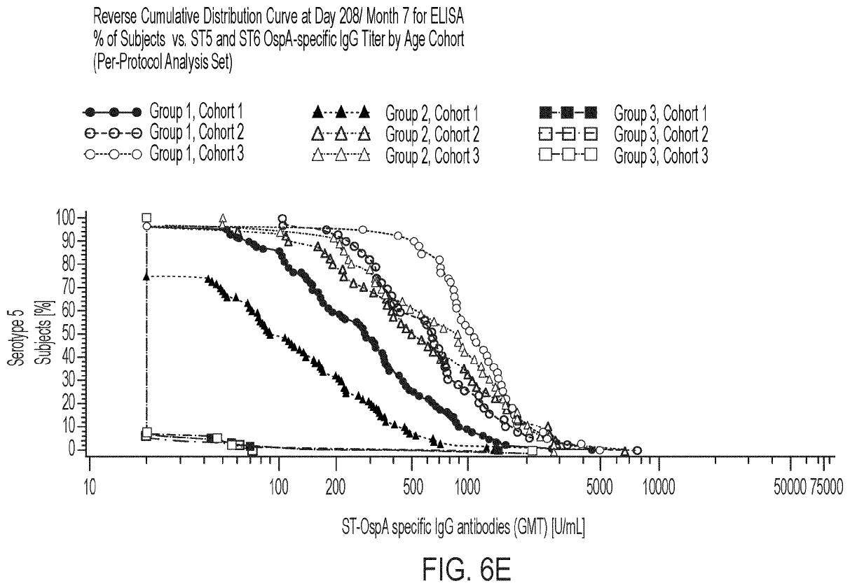

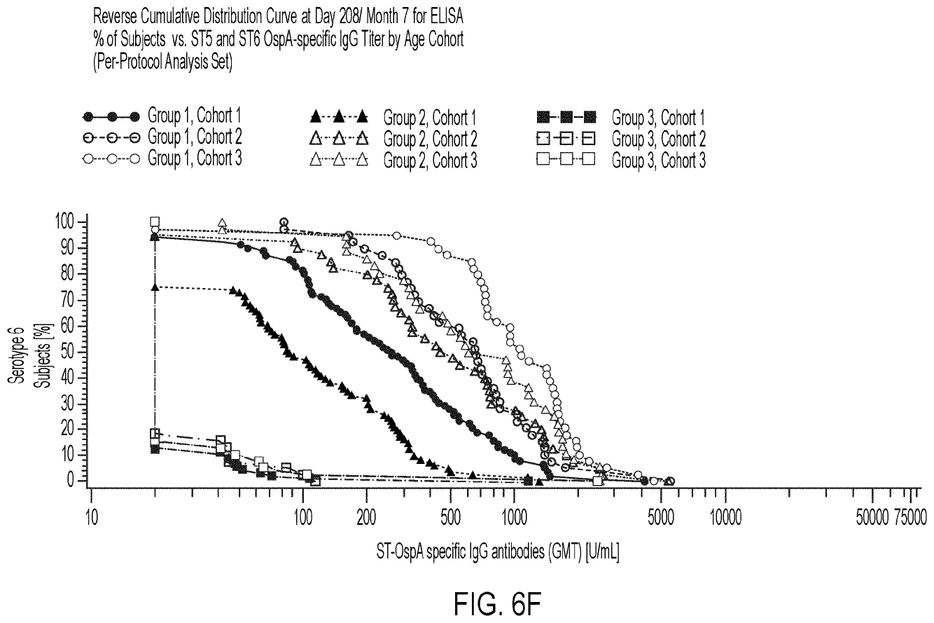

FIGS. 6A-6F show the reverse cumulative distribution curve at Day 208/Month 7 for ELISA % of subjects vs. A-B) ST1 and ST2, C-D) ST3 and ST4, E-F) ST5 and ST6, OspA-specific IgG titer by age cohort.

Table 5 shows the Geometric Mean Fold Rises (GMFRs) compared to Day 0 for OspA-specific IgG on Day 208/Month 7. Overall, GMFRs ranged from 15.6 [ST1] to 31.5 [ST2] in the M 0-2-6 schedule group and from 9.6 [ST1] to 22.8 [ST2] in the M 0-6 schedule group. In adults (18-65 years), GMFRs ranged from 9.2 [ST1] to 19.7 [ST2] in the M 0-2-6 schedule group and from 4.0 [ST1] to 10.6 [ST2] in the M 0-6 schedule group. In adolescents (12-17 years), GMFRs ranged from 25.7 [ST1] to 45.3 [ST2] in the M 0-2-6 schedule group and from 19.7 [ST6] to 46.9 [ST3] in the M 0-6 schedule group. In children, GMFRs ranged from 32.3 [ST1] to 66.9 [ST2] in the M 0-2-6 schedule group and from 25.4 [ST1] to 64.7 [ST3] in the M 0-6 schedule group.

TABLE 5
GMFRs compared to Day 0 for OspA-specific IgG on Day 208/Month 7.

| | | 180 ug MBV | | | |
|---|---|---|---|---|---|
| | | Group 1 | Group 2 | | |
| | | M 0-2-6 | M 0-6 | Placebo | |
| | | GMFR [95% CI] | GMFR [95% CI] | GMFR [95% CI] | |
| | | N(5-65 yrs) = 177 | N(5-65 yrs) = 162 | N(5-65 yrs) = 177 | |
| | | N(18-65 yrs) = 96 | N(18-65 yrs) = 84 | N(18-65 yrs) = 94 | |
| | | N(12-17 yrs) = 41 | N(12-17 yrs) = 40 | N(12-17 yrs) = 42 | p-value |
| Serotype | Age | N(5-11 yrs) = 40 | N(5-11 yrs) = 38 | N(5-11 yrs) = 41 | overall |
| ST1 | 5-65 yrs | 15.6 [12.8, 19.0] | 9.6 [7.6, 12.2] | 1.1 [1.0, 1.2] | <.0001 |
| | 18-65 yrs | 9.2 [7.1, 12.1] | 4.0 [3.1, 5.3] | 1.1 [1.0, 1.3] | <.0001 |
| | 12-17 yrs | 25.7 [18.2, 36.2] | 23.4 [15.7, 34.8] | 1.1 [1.0, 1.2] | <.0001 |
| | 5-11 yrs | 32.3 [24.3, 42.9] | 25.4 [17.4, 37.1] | 1.0 [0.8, 1.2] | <.0001 |
| ST2 | 5-65 yrs | 31.5 [26.7, 37.2] | 22.8 [18.4, 28.4] | 1.0 [1.0, 1.1] | <.0001 |
| | 18-65 yrs | 19.7 [15.8, 24.5] | 10.6 [8.1, 13.7] | 1.0 [0.9, 1.1] | <.0001 |
| | 12-17 yrs | 45.0 [34.6, 58.4] | 45.3 [31.0, 66.4] | 1.0 [1.0, 1.1] | <.0001 |
| | 5-11 yrs | 66.9 [50.5, 88.6] | 60.1 [44.2, 81.9] | 1.0 [0.8, 1.2] | <.0001 |
| ST3 | 5-65 yrs | 28.3 [23.9, 33.6] | 21.5 [17.0, 27.2] | 1.0 [1.0, 1.1] | <.0001 |
| | 18-65 yrs | 17.7 [14.2, 22.1] | 9.0 [6.8, 11.8] | 1.1 [1.0, 1.2] | <.0001 |
| | 12-17 yrs | 44.2 [32.7, 59.8] | 46.9 [31.3, 70.2] | 1.0 [1.0, 1.1] | <.0001 |
| | 5-11 yrs | 54.7 [40.5, 74.0] | 64.7 [46.4, 90.1] | 1.0 [0.8, 1.2] | <.0001 |
| ST4 | 5-65 yrs | 20.1 [16.8, 24.1] | 11.9 [9.5, 14.9] | 1.1 [1.0, 1.2] | <.0001 |
| | 18-65 yrs | 11.8 [9.3, 15.0] | 5.2 [4.0, 6.7] | 1.1 [1.0, 1.2] | <.0001 |
| | 12-17 yrs | 32.2 [24.0, 43.3] | 26.1 [17.7, 38.4] | 1.1 [1.0, 1.2] | <.0001 |
| | 5-11 yrs | 43.9 [33.0, 58.6] | 32.7 [23.1, 46.2] | 1.0 [0.8, 1.3] | <.0001 |
| ST5 | 5-65 yrs | 21.3 [17.9, 25.3] | 11.7 [9.3, 14.7] | 1.1 [1.0, 1.2] | <.0001 |
| | 18-65 yrs | 12.6 [10.1, 15.7] | 4.9 [3.8, 6.4] | 1.1 [1.0, 1.2] | <.0001 |
| | 12-17 yrs | 31.4 [23.9, 41.3] | 25.8 [17.7, 37.7] | 1.0 [0.9, 1.2] | <.0001 |
| | 5-11 yrs | 49.6 [37.3, 65.9] | 34.1 [24.4, 47.5] | 1.1 [0.9, 1.3] | <.0001 |
| ST6 | 5-65 yrs | 19.0 [15.8, 22.8] | 10.3 [8.3, 12.9] | 1.1 [1.0, 1.2] | <.0001 |
| | 18-65 yrs | 11.4 [9.0, 14.5] | 4.6 [3.6, 5.9] | 1.1 [1.0, 1.2] | <.0001 |
| | 12-17 yrs | 29.9 [22.4, 39.8] | 19.7 [13.4, 28.8] | 1.1 [1.0, 1.3] | <.0001 |
| | 5-11 yrs | 38.8 [26.9, 56.0] | 31.1 [21.9, 44.1] | 1.1 [0.9, 1.4] | <.0001 |

In adults, GMFRs for all six serotypes were statistically significantly higher in the MO-2-6 schedule group vs. the M 0-6 schedule group, see Table 6.

TABLE 6
Statistical significance in GMFRs

| Statistical | | |
|---|---|---|
| significant | | |
| comparison | GM ratio | |
| between MBV | estimate | |
| groups | [95% CI] | p-value |
| Study Group 1 vs. | 18-65 yrs: | ST1, |
| Study Group 2 | ST1: 2.3 [1.6, 3.4]; | ST2: <.0001 |
| | ST2: 1.9 [1.3, 2.6] | |
| Study Group 1 vs. | 18-65 yrs: | ST3, |
| Study Group 2 | ST3: 2.0 [1.4, 2.8]; | ST4: <.0001 |
| | ST4: 2.3 [1.6, 3.21 | |
| Study Group 1 vs. | 18-65 yrs: | ST5, |
| Study Group 2 | ST5: 2.6 [1.8, 3.6]; | ST6: <.0001 |
| | ST6: 2.5 [1.8, 3.51 | |

FIG. 7 shows the seroconversion rates (SCRs) on Day 208/Month 7. Seroconversion rate is defined as seroconversion from seronegative to seropositive or ≥four-fold increase in IgG titer compared to baseline if subject was tested OspA seropositive at baseline. Overall, SCRs ranged from 92.8% [ST1] to 98.8% [ST3] in the M 0-2-6 schedule group and from 83.4% [ST1] to 94.3% [ST2] in the M 0-6 schedule group. In adults (18-65 years), SCRs ranged from 88.9% [ST1] to 98.9% [ST3] in the M 0-2-6 schedule group and from 70.4% [ST1] to 90.1% [ST2] in the M 0-6 schedule group. In adolescents (12-17 years), SCRs ranged from 97.4% [ST1] to 100% [ST2/3/4/5/6] in the M 0-2-6 schedule group and from 95.0% [ST6] to 97.5% [ST1/2/3/4/5] in the M 0-6 schedule group. In children (5-11 years), SCRs ranged from 94.9% [ST6] to 97.4% [ST1/ST2/3/4/5] in the M 0-2-6 schedule group and from 97.2% [ST1] to 100% [ST2/3/4/5/6] in the M 0-6 schedule group.

### Immunogenicity Analysis: Day 365/Month 12 (6 Months Post Priming)

An immunogenicity assessment was performed on samples collected at Visit 7, Day 365/Month 12, including GMTs (Geometric Mean Titers), GMFRs (Geometric Mean Fold Rises) and SCRs (Seroconversion Rates) for IgG antibodies against each OspA serotype (ST1 to ST6). Higher antibody levels were observed in the three-dose Month 0-2-6 vaccination schedule versus the two-dose Month 0-6 vaccination schedule. Antibody levels remained above baseline six months after completion of a three-dose (Month 0-2-6) or a two-dose (Month 0-6) vaccination schedule GMFRs compared to baseline were 1.9-fold for Serotype 1 (ST1) to 3.2-fold Serotype 2 (ST2) across all age groups in the Month 0-2-6 vaccination schedule. The highest GMFRs were reported in the 5 to 11 years old age group, with GMFR levels at 2.8-fold (ST1) to 6.6-fold (ST2).

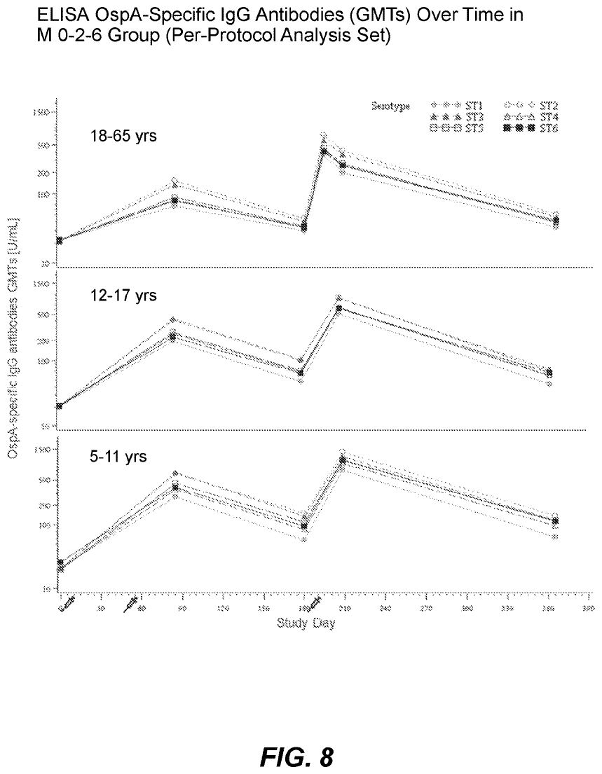

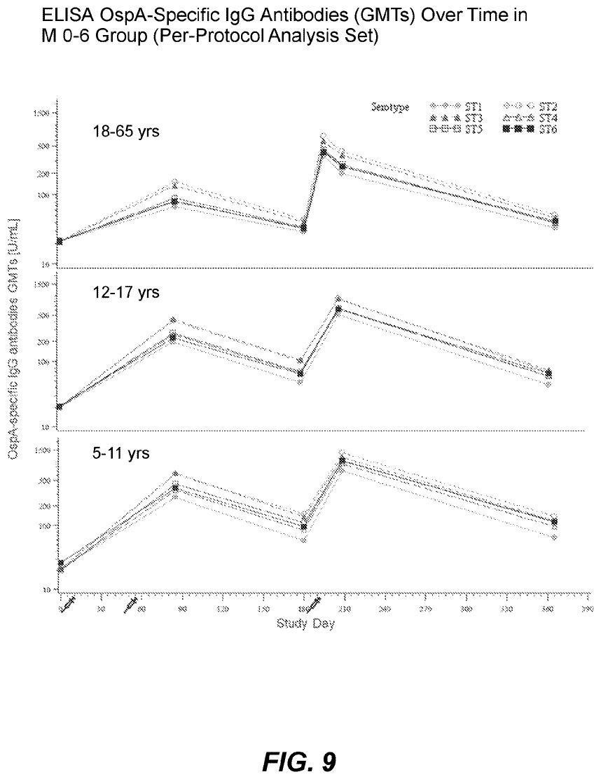

Human sera was analyzed for IgG against each OspA serotype (ST1 to ST6) separately by a quantitative ELISA. Table 7 shows the GMTs for OspA-specific IgG by serotype on Day 365/Month 12 in the M 0-2-6 schedule group. Table 8 shows the GMTs for OspA-specific IgG by serotype on Day 365/Month 12 in the M 0-6 schedule group. FIGS. 8 and 9 show the GMTs for OspA-specific IgG by serotype over time in the M 0-2-6 and M 0-6 schedule groups, respectively.

TABLE 7
GMTs for OspA-specific IgG by Serotype on Day 365/Month 12 in M 0-2-6

| | Day 365/ Month 12 | | Day 365/ Month 12 | | Day 365/ Month 12 | |
|---|---|---|---|---|---|---|
| | N(18-65 yrs) = 96 | | N(12-17 yrs) = 41 | | N(5-11 yrs) = 40 | |
| | GMTs | | GMTs | | GMTs | |
| | [U/mL] | [95% CI] | [U/mL] | [95% CI] | [U/mL] | [95% CI] |
| ST1 | 33.4 | [27.8, 40.0] | 44.0 | [32.2, 60.1] | 63.4 | [46.7, 86.1] |
| ST2 | 50.9 | [42.3, 61.4] | 68.8 | [48.5, 97.4] | 138.9 | [107.7, 179.2] |
| ST3 | 46.6 | [38.6, 56.3] | 73.7 | [51.7, 105.1] | 118.6 | [85.0, 165.5] |
| ST4 | 39.9 | [33.5, 47.6] | 58.7 | [43.1, 80.1] | 96.1 | [71.8, 128.4] |
| ST5 | 39.2 | [32.9, 46.7] | 59.7 | [42.8, 83.2] | 116.6 | [87.9, 154.7] |
| ST6 | 41.4 | [34.5, 49.7] | 65.7 | [47.3, 91.3] | 113.1 | [84.7, 151.1] |

TABLE 8
GMTs for OspA-specific IgG by Serotype on Day 365/Month 12 in M 0-6

| | Day 365/ Month 12 | | Day 365/ Month 12 | | Day 365/ Month 12 | |
|---|---|---|---|---|---|---|
| | N(18-65 yrs) = 84 | | N(12-17 yrs) = 40 | | N(5-11 yrs) = 38 | |
| | GMTs | | GMTs | | GMTs | |
| | [U/mL] | [95% CI] | [U/mL] | [95% CI] | [U/mL] | [95% CI] |
| ST1 | 23.1 | [21.0, 25.4] | 36.5 | [28.7, 46.4] | 44.3 | [33.5, 58.6] |
| ST2 | 35.9 | [30.6, 42.3] | 72.8 | [53.6, 98.8] | 98.0 | [73.2, 131.2] |
| ST3 | 34.6 | [29.4, 40.6] | 86.1 | [63.4, 116.8] | 120.6 | [91.1, 159.6] |
| ST4 | 25.9 | [23.1, 29.0] | 52.4 | [40.0, 68.6] | 58.0 | [46.0, 73.1] |
| ST5 | 27.2 | [24.0, 30.7] | 56.4 | [43.1, 73.8] | 55.1 | [41.6, 73.0] |
| ST6 | 26.2 | [23.4, 29.2] | 51.2 | [39.4, 66.5] | 64.0 | [50.3, 81.4] |

Table 9 shows the estimated percent (%) of subjects above the 125 U/mL ELISA threshold for ST1 at Day 208/Month 7 and Day 365/Month 12 in the M 0-2-6 (Group 1) and M 0-6 (Group 2) schedule groups.

TABLE 9
Estimated % of subjects above ELISA ST1 threshold of 125 U/mL

| | | | Estimated % of subjects | |
|---|---|---|---|---|
| | | | above threshold of | |
| | | | 125 U/mL at: | |
| ELISA ST1 | | | Day 208/ | Day 365/ |
| Threshold | Schedule | Age | Month 7 | Month 12 |
| 125 U/mL | M 0-2-6 | 18-65 yrs | 70% | 8% |
| | | 12-17 yrs | 98% | 18% |
| | | 5-11 yrs | 98% | 20% |
| | M 0-6 | 18-65 yrs | 40% | 5% |
| | | 12-17 yrs | 88% | 10% |
| | | 5-11 yrs | 95% | 15% |

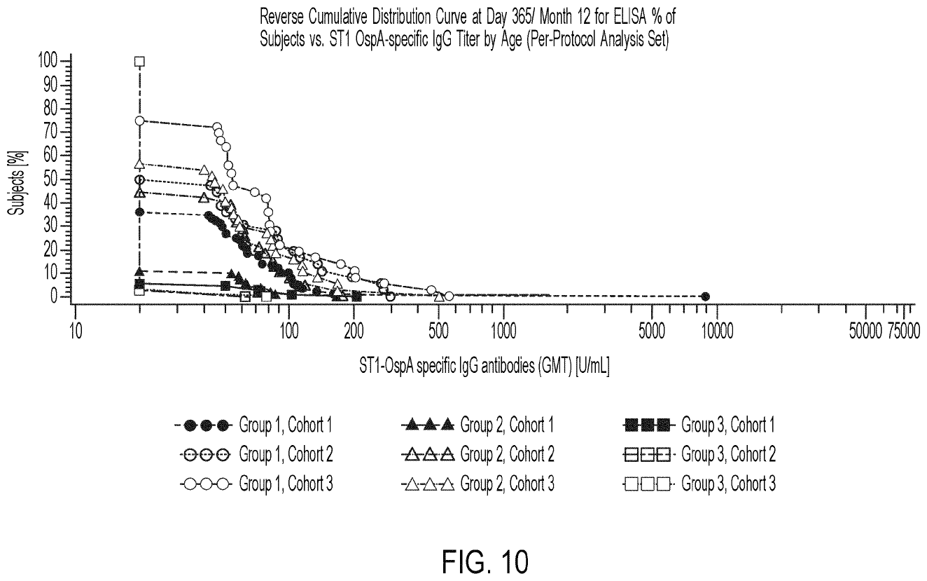

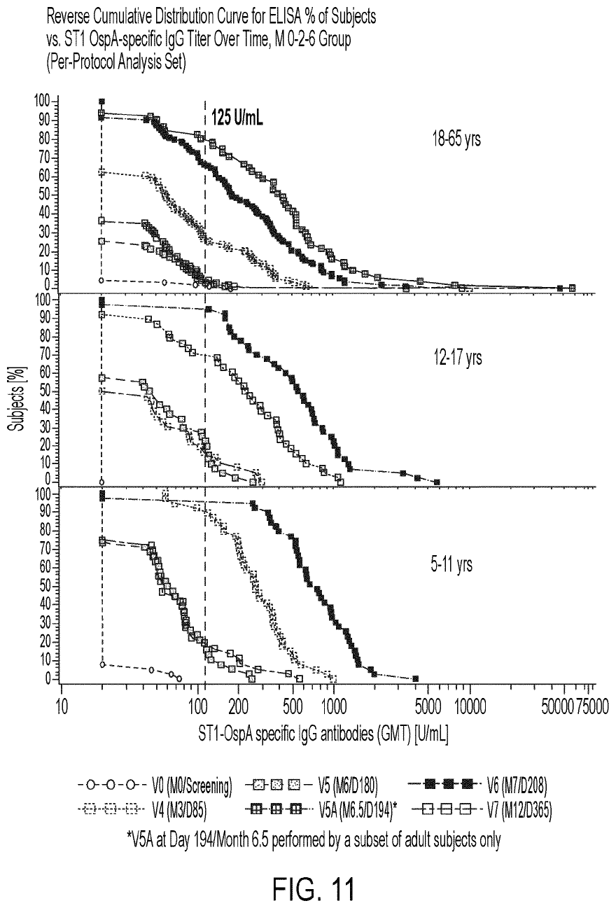

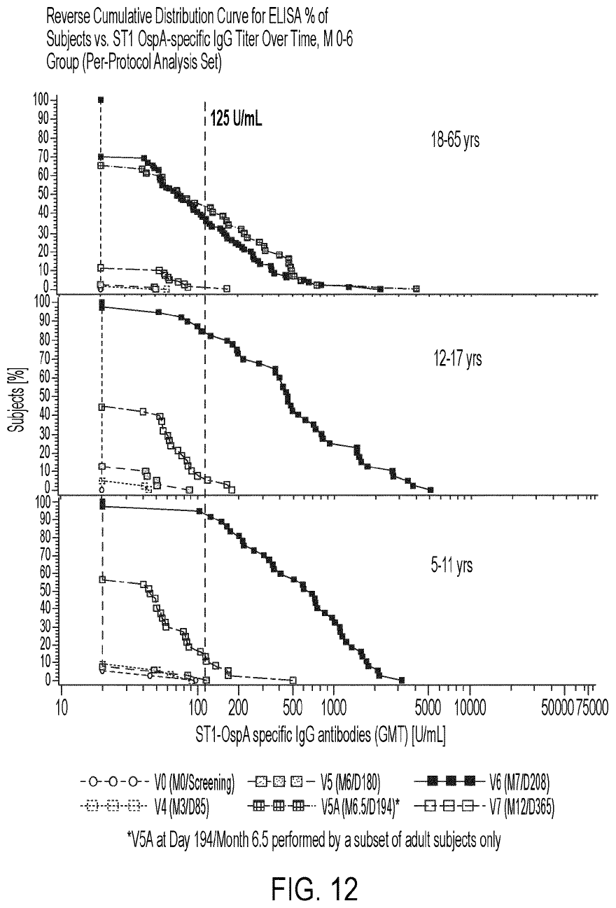

FIG. 10 shows the reverse cumulative distribution curve at Day 365/Month 12 for ELISA % of subjects vs ST1 OspA-specific IgG titer by age. FIG. 11 shows the reverse cumulative distribution curve for ELISA % of subjects vs ST1 OspA-specific IgG titer over time for M 0-2-6 schedule group. FIG. 12 shows the reverse cumulative distribution curve for ELISA % of subjects vs ST1 OspA-specific IgG titer over time for M 0-6 schedule group.

Table 10 shows the GMFRs as compared to Day 0 for ELISA OspA-specific IgG for subjects ages 5-65 years over time.

TABLE 10
GMFRs compared to Day 0 for OspA-specific IgG for subjects 5-65 years.

| | Study | N | ST1 | ST2 | ST3 | ST4 | ST5 | ST6 |
|---|---|---|---|---|---|---|---|---|
| | Group | (5-65 yrs) | [95% CI] | [95% CI] | [95% CI] | [95% CI] | [95% CI] | [95% CI] |
| Day 85/ | M 0-2-6 | N = 177 | 5.5 | 12.6 | 12.1 | 7.0 | 7.9 | 6.4 |
| Month 3 | | | [4.5, 6.6] | [10.4, 15.2] | [9.9, 14.7] | [5.8, 8.4] | [6.5, 9.5] | [5.4, 7.7] |
| | M 0-6 | N = 162 | 1.0 | 1.1 | 1.1 | 1.1 | 1.0 | 1.1 |
| | | | [1.0, 1.0] | 1.0, 1.2] | [1.1, 1.2] | [1.0, 1.1] | [1.0, 1.1] | [1.0, 1.1] |
| Day 180/ | M 0-2-6 | N = 177 | 1.8 | 3.3 | 3.1 | 2.3 | 2.5 | 2.2 |
| Month 6 | | | [1.6, 2.0] | [2.9, 3.9] | [2.7, 3.7] | [2.0, 2.5] | [2.2, 2.8] | [1.9, 2.5] |
| | M 0-6 | N = 162 | 1.1 | 1.1 | 1.1 | 1.1 | 1.1 | 1.1 |
| | | | [1.0, 1.1] | 1.0, 1.2] | [1.0, 1.2] | [1.0, 1.1] | [1.0, 1.1] | [1.0, 1.1] |
| Day 208/ | M 0-2-6 | N = 177 | 15.6 | 31.5 | 28.2 | 20.1 | 21.3 | 19.1 |
| Month 7 | | | [12.9, 18.9] | [26.7, 37.0] | [23.9, 33.4] | [16.9, 24.0] | [18.0, 25.3] | [15.9, 22.8] |
| | M 0-6 | N = 162 | 9.7 | 23.0 | 21.7 | 12.0 | 11.8 | 10.4 |
| | | | [7.7, 12.3] | [18.5, 28.6] | [17.1, 27.4] | [9.6, 15.0] | [9.4, 14.8] | [8.3, 13.0] |
| Day 365/ | M 0-2-6 | N = 177 | 1.9 | 3.2 | 3.0 | 2.5 | 2.6 | 2.5 |
| Month 12 | | | [1.7, 2.2] | [2.8, 3.8] | [2.6, 3.5] | [2.2, 2.9] | [2.3, 3.0] | [2.2, 2.9] |
| | M 0-6 | N = 162 | 1.5 | 2.7 | 2.9 | 1.8 | 1.9 | 1.8 |
| | | | [1.3, 1.7] | [2.3, 3.1] | [2.5, 3.4] | [1.6, 2.1] | [1.7, 2.1] | [1.6, 2.0] |

Table 11 shows the GMFRs compared to Day 0 for ELISA OspA-specific IgG for subjects ages 5-11 years over time.

TABLE 11
GMFRs compared to Day 0 for OspA-specific IgG for subjects 5-11 years.

| | Study | N | ST1 | ST2 | ST3 | ST4 | ST5 | ST6 |
|---|---|---|---|---|---|---|---|---|
| | Group | (5-11 yrs) | [95% CI] | [95% CI] | [95% CI] | [95% CI] | [95% CI] | [95% CI] |
| Day 85/ | M 0-2-6 | N = 40 | 12.3 | 30.2 | 30.7 | 16.9 | 21.7 | 14.4 |
| Month 3 | | | [9.8, 15.3] | [23.4, 39.0] | [23.1, 40.8] | [13.0, 22.1] | [16.6, 28.4] | [10.9, 19.0] |
| | M 0-6 | N = 38 | 1.0 | 1.2 | 1.2 | 1.1 | 1.1 | 1.1 |
| | | | [1.0, 1.1] | [1.0, 1.3] | [1.0, 1.4] | [1.0, 1.3] | [1.0, 1.2] | [1.0, 1.2] |
| Day 180/ | M 0-2-6 | N = 40 | 2.5 | 7.1 | 6.4 | 4.0 | 5.2 | 3.8 |
| Month 6 | | | [2.0, 3.2] | [5.6, 9.1] | [4.7, 8.5] | [3.1, 5.1] | [4.1, 6.8] | [2.9, 5.0] |
| | M 0-6 | N = 38 | 1.0 | 1.1 | 1.1 | 1.1 | 1.1 | 1.2 |
| | | | [1.0, 1.1] | [1.0, 1.2] | [1.0, 1.2] | [1.0, 1.2] | [1.0, 1.2] | [1.0, 1.3] |
| Day 208/ | M 0-2-6 | N = 40 | 32.3 | 66.9 | 54.7 | 43.9 | 49.6 | 38.8 |
| Month 7 | | | [24.3, 42.9] | [50.5, 88.6] | [40.5, 74.0] | [33.0, 58.6] | [37.3, 65.9] | [26.9, 56.0] |
| | M 0-6 | N = 38 | 25.7 | 60.7 | 65.1 | 33.1 | 34.5 | 31.5 |
| | | | [17.8, 37.1] | [44.9, 81.9] | [47.1, 89.9] | [23.7, 46.4] | [24.9, 47.7] | [22.4, 44.3] |
| Day 365/ | M 0-2-6 | N = 40 | 2.8 | 6.6 | 5.4 | 4.4 | 5.5 | 4.3 |
| Month 12 | | | [2.0, 3.8] | [5.1, 8.6] | [3.9, 7.6] | [3.3, 5.9] | [4.2, 7.3] | [3.3, 5.7] |
| | M 0-6 | N = 38 | 2.1 | 4.9 | 6.0 | 2.9 | 2.8 | 3.1 |
| | | | [1.6, 2.7] | [3.7, 6.6] | [4.6, 8.0] | [2.3, 3.7] | [2.1, 3.7] | [2.4, 3.9] |

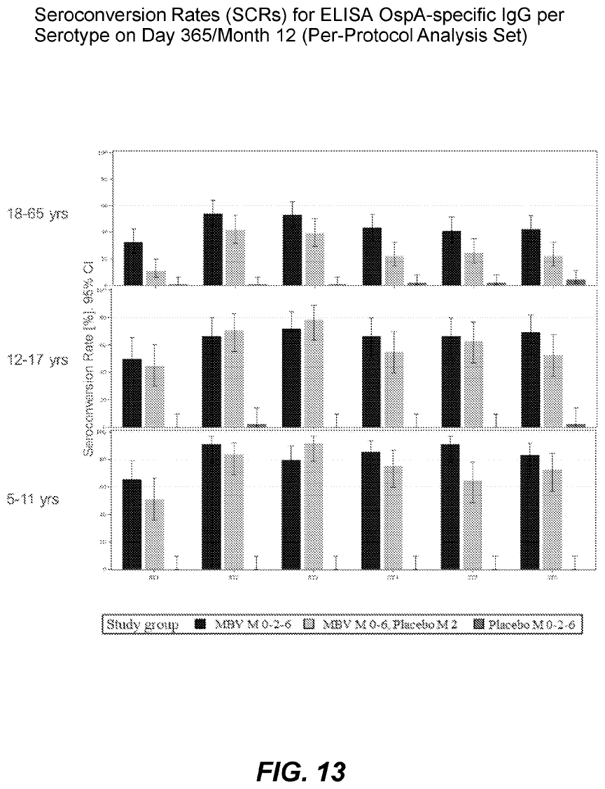

FIG. 13 shows the seroconversion rates (SCRs) for ELISA OspA-specific IgG per serotype on Day 365/Month 12. Seroconversion rate is defined as seroconversion from seronegative to seropositive or ≥four-fold increase in IgG titer compared to baseline if subject was tested OspA seropositive at baseline.

### Example 2: A Phase 3, Multicenter, Placebo-Controlled, Randomized, Observer-Blinded Trial to Evaluate the Efficacy, Safety, Tolerability, Immunogenicity, and Lot Consistency of a 6-Valent OspA-Based Lyme Disease Vaccine in Healthy Participants 25 Years of Age (VALOR)

A 6-valent OspA-based Lyme disease vaccine (multivalent Borrelia vaccine (MBV)) is being investigated for active immunization for the prevention of Lyme disease caused by Borrelia species (OspA serotypes 1, 2, 3, 4, 5, and 6) in individuals 5 years of age and older.

### Overall Design

The purpose of the study is to establish the efficacy, safety, tolerability, and immunogenicity of MBV for prevention of Lyme disease within North America (NA) and Europe during the Lyme disease season following the primary vaccination series and/or following the primary series and booster dose during the subsequent Lyme disease season. This study also serves as a lot-consistency study, for operational efficiency, based on the immunogenicity evaluation of 3 independent lots in a subset of participants.

Healthy participants ≥5 years of age will be recruited from areas with high levels of endemic Lyme disease and randomized to receive MBV or placebo (saline). This study is planned to be conducted in regions of NA and Europe where Lyme disease is endemic and prevalent to ensure appropriate geographic representation. Site-based randomization will be used to assign individuals to different groups. Randomization will be stratified by geographic region (Europe and US) and age (5-11 years, 12-17 years, 18-44 years, 45-64 years, and >65 years). US participants will be randomized to receive MBV from 3 different lots or placebo, with a randomization ratio of 1:1:1:3 (MBV from Lot 1: MBV from Lot 2: MBV from Lot 3: placebo) for each region/age stratum. European participants will be randomized in a 1:1 ratio to receive either MBV from Lot 1 or placebo. The overall study randomization ratio between MBV and placebo remains 1:1.

Comparison will be made between the Lyme disease cases of people receiving the study vaccine to those of the people who are not. This will help determine if the study vaccine is safe and effective. If enrolled, participants will need to visit the research site at least 7 times during the study. There will also be at least 1 telephone contact. It is expected that each participant will take part in this study for up to about 2 and a half years (30 months).

### Dosage and Schedule

Participants receive a 3-dose primary vaccination series at about 0, 2, and 5 to 9 months and then receive a booster dose about 12 months later. Participants receive 1 dose of MBV (180 μg) or placebo (1:1 ratio) at each vaccination visit. MBV or placebo is administered intramuscularly by injecting 0.5 mL into the deltoid muscle, preferably of the nondominant arm, see Table 12.

TABLE 12
Study interventions administered.

| Arms | Assigned Intervention |
|---|---|
| Experimental: MBV Lot 1 (3-dose primary | Biological/Vaccine: MBV |
| vaccination series and booster dose) | Shot in the deltoid muscle |
| Shot in the deltoid muscle (preferable in | (preferable in the |
| the nondominant arm) | nondominant arm) |
| Experimental: MBV Lot 2 (3-dose primary | Biological/Vaccine: MBV |
| vaccination series and booster dose) | Shot in the deltoid muscle |
| Shot in the deltoid muscle (preferable in | (preferable in the |
| the nondominant arm) | nondominant arm) |
| Experimental: MBV Lot 3 (3-dose primary | Biological/Vaccine: MBV |
| vaccination series and booster dose) | Shot in the deltoid muscle |
| Shot in the deltoid muscle (preferable in | (preferable in the |
| the nondominant arm) | nondominant arm) |
| Placebo Comparator: Placebo (3-dose | Other: Saline |
| primary vaccination series and booster | Shot in the deltoid muscle |
| dose) | (preferable in the |
| Shot in the deltoid muscle (preferable in | nondominant arm) |
| the nondominant arm) | |

This study is designed to demonstrate whether a 3-dose extended-schedule primary vaccination series administered over 5 to 9 months and then a booster dose about one year (12 months) after completing the primary series, just prior to the second season, will protect against Lyme disease during the second Lyme disease season. The study is further designed to demonstrate whether the primary series will protect against Lyme disease during the first Lyme disease season. Vaccination of participants will occur at a time of year such that the primary series is completed from approximately April to early May before the peak Lyme disease season in Europe or US. The start of the primary series will occur from August to early December, with the majority of participants receiving the first dose of the primary series from mid-September to early November. The primary series will be followed by a booster dose 1 year later, from approximately March to early May just prior to the beginning of the second Lyme disease season.

### Objectives:

To demonstrate the efficacy of MBV in preventing confirmed Lyme disease in the Lyme disease season after completion of the primary series vaccination and booster dose.
To describe the safety profile of MBV as measured by the percentage of participants reporting local reactions, systemic events, adverse events (AEs), newly diagnosed chronic medical conditions (NDCMCs), and serious adverse events (SAEs).
To demonstrate that the immune responses to the 6 serotypes induced by MBV are equivalent across 3 independent lots.
To demonstrate the efficacy of MBV in preventing confirmed Lyme disease in the Lyme disease season after completion of the primary series vaccination.
To describe the efficacy of MBV in reducing otherwise undiagnosed Lyme disease seroconversion after each season.
To describe the efficacy of MBV in preventing confirmed Lyme disease by geographic region (NA and Europe separately).

### Outcome Measures:

Relative risk reduction of confirmed Lyme disease cases in the MBV group compared to the placebo group [Time Frame: In the Lyme disease season beginning 1 month after receiving the booster dose (28 days after completion of the booster dose until 31 October)]
   Clinically- and laboratory-confirmed Lyme disease caused by B burgdorferi sensu lato as confirmed by the Endpoint Adjudication Committee
Percentage of participants reporting prompted local reactions [Time Frame: Within 7 days following each study intervention administration]
Percentage of participants reporting prompted systemic events [Time Frame: Within 7 days following each study intervention administration]
Percentage of participants reporting adverse events (AEs) [Time Frame: Through 1 month following each study intervention administration]
Percentage of participants reporting newly diagnosed chronic medical conditions (NDCMCs) [Time Frame: Through the end-of-study visit]
Percentage of participants reporting serious adverse events (SAEs) [Time Frame: Through the end-of-study visit]
Geometric mean ratio (GMR) of ant-OspA titers for each serotype (ST1-ST6) for Lot 1 to Lot 2 [Time Frame: At 1 month after completion of the primary series]
Geometric mean ratio (GMR) of anti-OspA titers for each serotype (ST1-ST6) for Lot 1 to Lot 3 [Time Frame: At 1 month after completion of the primary series]
Geometric mean ratio (GMR) of anti-OspA titers for each serotype (ST1-T6) for Lot 2 to Lot 3 [Time Frame: At 1 month after completion of the primary series]
Relative risk reduction of confirmed Lyme disease cases in the MBV group compared to the placebo group [Time Frame: In the Lyme disease season beginning 1 month after completing the primary series (28 days after completion of the primary series until 31 October)]
   Clinically- and laboratory-confirmed Lyme disease caused by B burgdorferi sensu lato as confirmed by the Endpoint Adjudication Committee
Relative risk reduction of Lyme disease-specific seroconversion in otherwise-undiagnosed Lyme disease cases in the MBV group compared to the placebo group [Time Frame: In the Lyme disease season beginning 1 month after completing the primary series (28 days after completion of the primary series until 31 October)]
   Nonvaccine-antigen seroconversion as measured at the central laboratory
Relative risk reduction of Lyme disease-specific seroconversion in otherwise-undiagnosed Lyme disease cases in the MBV group compared to the placebo group [Time Frame: In the Lyme disease season beginning 1 month after receiving the booster dose (28 days after completion of the booster dose until 31 October)]
   Nonvaccine-antigen seroconversion as measured at the central laboratory
Vaccine efficacy among participants enrolled from North American sites [Time Frame: In the Lyme disease season beginning 1 month after completing the primary series (28 days after completion of the primary series until 31 October)]
   Clinically- and laboratory-confirmed Lyme disease caused by B burgdorferi sensu lato as confirmed by the Endpoint Adjudication Committee
Vaccine efficacy among participants enrolled from European sites [Time Frame: In the Lyme disease season beginning 1 month after completing the primary series (28 days after completion of the primary series until 31 October)]
   Clinically- and laboratory-confirmed Lyme disease caused by B burgdorferi sensu late as confirmed by the Endpoint Adjudication Committee
Vaccine efficacy among participants enrolled from North American sites [Time Frame: In the Lyme disease season beginning 1 month after receiving the booster dose (28 days after completion of the booster dose until 31 October)]
   Clinically- and laboratory-confirmed Lyme disease caused by B burgdorferi sensu late as confirmed by the Endpoint Adjudication Committee
Vaccine efficacy among participants enrolled from European sites [Time Frame: In the Lyme disease season beginning 1 month after receiving the booster dose (28 days after completion of the booster dose until 31 October)]
   Clinically- and laboratory-confirmed Lyme disease caused by B burgdorferi sensu lato as confirmed by the Endpoint Adjudication Committee

### Study Population

### Key Inclusion Criteria:

Male or female participants 5 years of age at enrollment in all countries where pediatric enrollment is permitted. In countries or sites where enrollment of children is not permitted, male or female participants 18 years of age at the time of informed consent.

Participants who reside in areas with endemic Lyme disease and who lead lifestyles that put them at increased risk for Lyme disease. For example, this could include, but not be limited to:
Individuals who work in B burgdorferi-infected/tick-infested areas, especially those with occupations that may be associated with higher risk of exposure, such as landscaping, forestry, and wildlife and parks management.
Individuals who pursue recreational activities such as hiking, camping, fishing, hunting, jogging, or gardening in such areas.
Individuals who live on land plots with tree lines and come into contact with these trees regularly.
Individuals who have dogs that regularly are outdoors and frequently return with attached ticks.
Individuals who participate in activities in areas with tall grass, smaller wooded areas beside forests, open fields, lakesides, and riversides.
Any other risk factors determined at the discretion of the investigators

### Key Exclusion Criteria:

Any diagnosis of Lyme disease within the past 3 months.
Any history of Lyme carditis, neuroborreliosis, arthritis, or other manifestations of disseminated Lyme disease regardless of when diagnosed.
Known tick bite within the past 4 weeks.
Newly developed or unstable underlying conditions that may interfere with the assessment of Lyme disease, including but not limited to chronic arthralgia/arthritis, second/third-degree AV heart block, chronic pain syndromes, and chronic skin conditions that reduce the ability to detect cutaneous manifestations of Lyme disease.
Any autoimmune condition with a manifestation (eg, arthritic and neurologic) that may interfere with the assessment of Lyme disease.
Chronic systemic doxycycline or minocycline or other tetracycline class drug use for acne or any other chronic suppressive antibiotics used to treat other conditions.

What is claimed is:

1. A method of eliciting an immune response protective against Lyme disease in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least three doses of the composition.

2. A method for treating, preventing or reducing the risk of Lyme disease in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least three doses of the composition.

3. A method of vaccinating a subject against Lyme disease, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least three doses of the composition.

4. The method of any one of claims 1 to 3, wherein a second dose is administered in a period of at least 6 weeks to at most 3 months after a first dose is administered, and a third dose is administered in a period of at least 5 months to at most 9 months or at least 5 months to at most 7 months after the first dose is administered.

5. The method of any one of claims 1 to 4, wherein a second dose is administered in a period of at least 50 days to at most 70 days after a first dose is administered, and a third dose is administered in a period of at least 170 days to at most 190 days after the first dose is administered.

6. The method of any one of claims 1 to 5, wherein a first dose is administered on day 1, a second dose is administered about 2 months after the first dose, and a third dose is administered about 6 months after the first dose is administered.

7. The method of any one of claims 1 to 6, wherein a fourth dose is administered in the period of at least 15 months to at most 21 months, particularly in the period of at least 17 months to at most 19 months after the first dose is administered.

8. The method of any one of claims 1 to 7, wherein a fourth dose is administered about 18 months after the first dose is administered or about 12 months after the third administration.

9. The method of any one of claims 1 to 8, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.

10. The method of any one of claims 1 to 9, wherein the subject is an adult subject, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.

11. The method of any one of claims 1 to 10, wherein the subject is a pediatric subject, wherein a first dose is administered on day 1, a second dose is administered in a period of at least 50 days to at most 70 days after a first dose, and a third dose is administered in a period of at least 170 days to at most 190 days after the first dose, and wherein optionally a fourth dose is administered about 18 months after the first dose is administered or about 12 months after the third administration.

12. The method of any one of claims 1 to 11, wherein a further dose is administered every year after the third dose or the optional fourth dose is administered, particularly after 1 year, after 2 years, and after 3 years.

13. A method of eliciting an immune response protective against Lyme disease in a subject comprising, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least two doses of the composition.

14. A method for treating, preventing or reducing the risk of Lyme disease in a subject, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least two doses of the composition.

15. A method of vaccinating a subject against Lyme disease, the method comprising administering to the subject a composition comprising a fusion protein of SEQ ID NO: 1 (Lip-S1 D1-S2D1), a fusion protein of SEQ ID NO: 2 (Lip-S4D1-S3hybD1), and a fusion protein of SEQ ID NO: 3 (Lip-S5D1-S6D1) at a total protein content of the three fusion proteins of 180 μg per dose, wherein the method comprises administering at least two doses of the composition.

16. The method of any one of claims 13 to 15, wherein a second dose is administered in a period of at least 5 months to at most 7 months after a first dose is administered.

17. The method of any one of claims 13 to 16, wherein a second dose is administered in a period of at least 170 days to at most 190 days after a first dose is administered.

18. The method of any one of claims 13 to 17, wherein a first dose is administered on day 1 and a second dose is administered about 6 months after the first dose is administered.

19. The method of any one of claims 13 to 18, wherein a third dose is administered in the period of at least 15 months to at most 21 months, particularly in the period of at least 17 months to at most 19 months after the first dose is administered.

20. The method of any one of claims 13 to 19, wherein a third dose is administered about 18 months after the first dose is administered.

21. The method of any one of claims 13 to 20, wherein a first dose is administered on day 1, a second dose is administered in a period of at least 170 days to at most 190 days after the first dose, and wherein optionally a third dose is administered about 18 months after the first dose is administered.

22. The method of any one of claims 13 to 21, wherein the subject is an adult subject, wherein a first dose is administered on day 1, a second dose is administered in a period of at least 170 days to at most 190 days after the first dose, and wherein optionally a third dose is administered about 18 months after the first dose is administered.

23. The method of any one of claims 13 to 22, wherein the subject is a pediatric subject, wherein a first dose is administered on day 1, a second dose is administered in a period of at least 170 days to at most 190 days after the first dose, and wherein optionally a third dose is administered about 18 months after the first dose is administered.

24. The method of any one of claims 13 to 23, wherein a further dose is administered every year after the second dose or the optional third dose is administered, particularly after 1 year, after 2 years, and after 3 years.

25. The method of any one of claims 1 to 24, wherein the subject is aged 5 years or older.

26. The method of any one of claims 1 to 25, wherein the subject is an adult subject aged 18 years or older, such as 18-65 years and/or 50 years or older.

27. The method of any one of claims 1 to 24, wherein the subject is a pediatric subject from birth to 17 years.

28. The method of claim 27, wherein the pediatric subject is aged 5-17 years, such as 5-11 years and/or 12-17 years.

29. The method of any one of claims 1 to 28, wherein the composition elicits an immune response comprising an anti-OspA serotype 1, an anti-OspA serotype 2, an anti-OspA serotype 3, an anti-OspA serotype 4, anti-OspA serotype 5 and/or an anti-OspA serotype 6 antibody response.

30. The method of any one of claims 1 to 29, wherein the composition elicits an immune response comprising antibodies against Borrelia serotypes 1, 2, 3, 4, 5, and 6.

31. The method of any one of claims 1 to 30, wherein the composition elicits an immune response comprising antibodies against Borrelia serotypes 1, 2, 3, 4, 5, and/or 6, wherein the immune response after administering at least three doses of the composition to an adult subject is higher than the immune response after administering at least two doses of the composition to an adult subject.

32. The method of claim 31, wherein geometric mean titers (GMTs) of antibodies against Borrelia serotypes after administering at least three doses of the composition to an adult subject are at least 2.0-fold higher than the GMTs after administering at least two doses of the composition to an adult subject.

33. The method of claim 32, wherein the GMTs of antibodies against Borrelia serotypes after administering at least three doses of the composition to an adult subject are about 2.0-fold to about 3.0-fold-higher than the GMTs after administering at least two doses of the composition to an adult subject.

34. The method of any one of claims 1 to 33, wherein the composition elicits an immune response comprising antibodies against Borrelia serotypes 1, 2, 3, 4, 5, and/or 6, wherein the immune response after administering a second dose of the composition to a pediatric subject is at least as high as or higher than the immune response after administering a third dose of the composition to an adult subject.

35. The method of any one of claims 1 to 34, wherein the composition elicits an immune response comprising antibodies against Borrelia serotypes 1, 2, 3, 4, 5, and/or 6, wherein the immune response after administering at least three doses of the composition to a pediatric subject is higher than the immune response after administering at least three doses of the composition to an adult subject.

36. The method of claim 35, wherein geometric mean titers (GMTs) of antibodies against Borrelia serotypes after administering at least three doses of the composition to a pediatric subject are at least 2.0-fold higher than the GMTs after administering at least three doses of the composition to an adult subject.

37. The method of claim 36, wherein the GMTs of antibodies against Borrelia serotypes after administering at least three doses of the composition to a pediatric subject aged 12-17 years are about 2.0-fold to about 3.0-fold higher that the GMTs after administering at least three doses of the composition to an adult subject.

38. The method of claim 36, wherein the GMTs of antibodies against Borrelia serotypes after administering at least three doses of the composition to a pediatric subject aged 5-11 years are about 3.0-fold to about 5.0-fold higher that the GMTs after administering at least three doses of the composition to an adult subject.

39. The method of any one of claims 1 to 38, wherein the composition elicits an immune response comprising antibodies against Borrelia serotypes 1, 2, 3, 4, 5, and/or 6, wherein the immune response after administering at least two doses of the composition to a pediatric subject is higher than the immune response after administering at least two doses of the composition to an adult subject.

40. The method of claim 39, wherein geometric mean titers (GMTs) of antibodies against Borrelia serotypes after administering at least two doses of the composition to a pediatric subject are at least 2.0-fold higher than the GMTs after administering at least two doses of the composition to an adult subject.

41. The method of claim 40, wherein GMTs of antibodies against Borrelia serotypes after administering at least two doses of the composition to a pediatric subject aged 12-17 years are about 4.0-fold to about 6.0-fold higher that the GMTs after administering at least two doses of the composition to an adult subject.

42. The method of claim 39, wherein the GMTs of antibodies against Borrelia serotypes after administering at least two doses of the composition to a pediatric subject aged 5-11 years are about 5.0-fold to about 8.0-fold higher than the GMTs after administering at least two doses of the composition to an adult subject.

43. The method of any one of claims 1 to 42, wherein the composition elicits an immune response comprising antibodies against Borrelia serotypes 1, 2, 3, 4, 5, and 6 that is sustained for at least about 60 days, for at least about 180 days, for at least about 365 days, or for at least about 540 days.

44. The method of claim 43, wherein the immune response is sustained above baseline for at least about 180 days after administering at least two doses or at least three doses of the composition.

45. The method of claim 44, wherein geometric mean fold rises (GMFRs) of antibodies against Borrelia serotypes at least 180 days after administering at least three doses of the composition to a pediatric subject aged 5-11 are at least 2.0-fold higher than the GMFRs at baseline.

46. The method of claim 45, wherein the GMFRs of antibodies against Borrelia serotypes at least 180 days after administering at least three doses of the composition to a pediatric subject aged 5-11 are about 2.0-fold to 7.0-fold higher than the GMFRs at baseline.

47. The method of any one of claims 1 to 46, wherein the composition is administered to a subject in a volume of 0.25 ml, 0.5 ml, or 1.0 ml.

48. The method of claim 47, wherein the composition is administered to a subject in a volume of 0.5 ml.

49. The method of any one of claims 1 to 48, wherein the three fusion proteins comprise at least 60%, preferably at least 70%, more preferably at least 80% of all proteins in the composition.

50. The method of any one of claims 1 to 49, wherein the composition comprises the three fusion proteins in a weight ratio of 1:1:1 (Lip-S1 D1-S2D1:Lip-S4D1-S3hybD1:Lip-S5D1-S6D1).

51. The method of any one of claims 1 to 50, wherein the composition comprises at least one of sodium phosphate, sodium chloride, sucrose and polysorbate 20.

52. The method of claim 51, wherein the sodium phosphate is present at a concentration between 5 mM and 50 mM, the sodium chloride is present at a concentration between 100 mM and 200 mM, the sucrose is present at a concentration between 2.5% and 10% and the polysorbate 20 is present at a concentration between 0.01% and 0.1%.

53. The method of any one of claims 1 to 52, wherein the composition comprises an adjuvant.

54. The method of claim 53, wherein the adjuvant comprises an aluminum adjuvant.

55. The method of any one of claims 1 to 54, wherein the composition comprises L-methionine.

56. The method of claim 55, wherein the L-methionine is present in a concentration of at least 10 mmol/l.
